(19) United States
(12) Patent Application Publication
(10) Pub. No.: US 2008/0306109 A1
(43) Pub. Date: Dec. 11, 2008

(54) Indolizine Derivatives as Ligands of the Crth2 Receptor

(76) Inventors: George Hynd, Essex (GB); Nicholas Charles Ray, Essex (GB); Harry Finch, Essex (GB); John Gary Montana, Hertfordshire (GB); Michael Colin Cramp, Essex (GB); Trevor Keith Harrison, Essex (GB); Rosa Arienzo, Essex (GB); Paul Blaney, Essex (GB); Yann Griffon, Essex (GB); David Middlemiss, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/065,963

(22) PCT Filed: Sep. 14, 2006
(86) PCT No.: PCT/GB2006/003394

§ 371 Date: Aug. 16, 2008

(30) Foreign Application Priority Data
Sep. 14, 2005 (GB) 0518783.6

(51) Int. Cl. A61K 31/44; C07D 455/02; A61P 43/00

(52) U.S. Cl. 514/306; 546/138

(57) ABSTRACT

Compounds of formula (I) are CRTH2 antagonists, useful in the treatment of, for example, asthma, chronic obstructive pulmonary disease, rhinitis, allergic airway syndrome, and allergic rhinobronchitis. Formula (I) wherein R₁, R₂. R₃ and R₄ each independently are hydrogen, C₁-C₆alkyl, fully or partially fluorinated C₁-C₆alkyl, halo, —S(O)ₙR₁₀, —SO₂N(R₁₀)₂, —N(R₁₀)₂, —C(O)N(R₁₀)₂, —NR₁₀C(O)R₉, —CO₂R₁₀, —C(O)R₉, —NO₂, —CN or —OR₁₁; wherein each R₉ is independently C₁-C₆alkyl, aryl, heteroaryl; R₁₀ is independently hydrogen, C₁-C₆alkyl, aryl, or heteroaryl; R₁₁ is hydrogen, C₁-C₆alkyl, fully or partially fluorinated C₁-C₆alkyl or a group —SO₂R₁₀; n is 0, 1 or 2; R₅ is C₁-C₆alkyl, fully or partially fluorinated C₁-C₆alkyl, C₁-C₆alkenyl, C₁-C₆alkynyl, optionally substituted aryl, or optionally substituted heteroaryl; R₆ is hydrogen, C₁-C₆alkyl or fully or partially fluorinated C₁-C₆alkyl; R₇ and R₈ are independently hydrogen or C₁-C₆alkyl, or R₇ and R₈ together with the atom to which they are attached form a cycloalkyl group; and X is —CHR₆—, —S(O)ₙ—, —C(O)—, —NR₆SO₂— or —SO₂NR₆- wherein n is 0, 1 or 2.

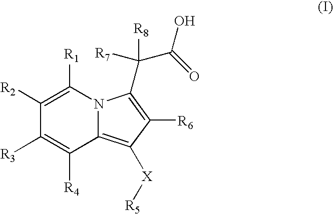

### FIELD OF THE INVENTION

This invention relates to a class of indolizine compounds which are ligands of the CRTH2 receptor (Chemoattractant Receptor-homologous molecule expressed on T Helper cells type 2), and their use in the treatment of diseases responsive to modulation of CRTH2 receptor activity, principally diseases having a significant inflammatory component. The invention also relates to novel members of that class of ligands and pharmaceutical compositions containing them.

### BACKGROUND TO THE INVENTION

Mast cells are known to play an important role in allergic and immune responses through the release of a number of mediators, such as histamine, leukotrienes, cytokines, prostaglandin D₂, etc (Boyce; Allergy Asthma Proc., 2004, 25, 27-30). Prostaglandin D₂ (PGD₂) is the major metabolite produced by the action of cyclooxygenase on arachidonic acid by mast cells in response to allergen challenge (Lewis et al; J. Immunol., 1982, 129, 1627-1631). It has been shown that PGD₂ production is increased in patients with systemic mastocytosis (Roberts; N. Engl. J. Med., 1980, 303, 1400-1404), allergic rhinitis (Naclerio et al; Am. Rev. Respir. Dis., 1983, 128, 597-602; Brown et al; Arch. Otolarynol. Head Neck Surg., 1987, 113, 179-183; Lebel et al; J. Allergy Clin. Immunol., 1988, 82, 869-877), bronchial asthma (Murray et al; N. Engl. J. Med., 1986, 315, 800-804; Liu et al; Am. Rev. Respir. Dis., 1990, 142, 126-132; Wenzel et al; J. Allergy Clin. Immunol., 1991, 87, 540-548), and urticaria (Heavey et al; J. Allergy Clin. Immunol., 1986, 78, 458-461). PGD₂ mediates it effects through two receptors, the PGD₂ (or DP) receptor (Boie et al; J. Biol. Chem., 1995, 270, 18910-18916) and the chemoattractant receptor-homologous molecule expressed on Th2 (or CRTH2) (Nagata et al; J. Immunol., 1999, 162, 1278-1289; Powell; Prostaglandins Luekot. Essent. Fatty Acids, 2003, 69, 179-185). Therefore, it has been postulated that agents that antagonize the effects of PGD₂ at its receptors may have beneficial effects in number of disease states.

The CRTH2 receptor has been shown to be expressed on cell types associated with allergic inflammation, such as basophils, eosinophils, and Th2-type immune helper cells (Hirai et al; J. Exp. Med., 2001, 193, 255-261). The CRTH2 receptor has been shown to mediate PGD₂-mediated cell migration in these cell types (Hirai et al; J. Exp. Med., 2001, 193, 255-261), and also to play a major role in neutrophil and eosinophil cell recruitment in a model of contact dermatitis (Takeshita et al; Int. Immunol., 2004, 16, 947-959). Ramatroban {(3R)-3-[(4-fluorophenyl)sulphonyl-amino]-1,2,3,4-tetrahydro-9H-carbazole-9-propanoic acid}, a dual CRTH2 and thromboxane A₂ receptor antagonist, has been shown to attenuate these responses (Sugimoto et al; J. Pharmacol. Exp. Ther., 2003, 305, 347-352; Takeshita et al; op. cit.). The potential of PGD₂ both to enhance allergic inflammation and induce an inflammatory response has been demonstrated in mice and rats. Transgenic mice over expressing PGD₂ synthase exhibit an enhanced pulmonary eosinophilia and increased levels of Th2 cytokines in response to allergen challenge (Fujitani et al, J. Immunol., 2002, 168, 443-449). In addition, exogenously administered CRTH2 agonists enhance the allergic response in sensitized mice (Spik et al; J. Immunol., 2005, 174, 3703-3708). In rats exogenously applied CRTH2 agonists cause a pulmonary eosinophilia but a DP agonist (BW 245C) or a TP agonist (I-BOP) showed no effect (Shirashi et al; J. Pharmacol. Exp Ther., 2005, 312, 954-960). These observations suggest that CRTH2 antagonists may have valuable properties for the treatment of diseases mediated by PGD₂.

In addition to ramatroban a number of other CRTH2 antagonists have been described. Examples include: indole-acetic acids (WO2003/022813; WO2003/066046; WO2003/066047; WO2003/097042; WO2003/097598; WO2003/101961; WO2003/101981; WO2004/007451; WO2004/078719; WO2004/106302; WO2005/019171; GB2407318; WO2005/040112; WO2005/040114; WO2005/044260); tetrahydroquinolines (EP1413306; EP1435356; WO2004/032848; WO2004/035543; WO2005/007094), and phenylacetic acids (WO2004/058164; WO2004/089884; WO2004/089885; WO2005/018529).

ES 421284 relates to indolizine derivatives which are claimed to be analgesic and anti-inflammatory agents. U.S. Pat. No. 6,645,976 discloses indolizines as sPLA2 inhibitors. DE 2046904 and GB 1174124 also relate to indolizine derivatives. Malonne, H. et al. Pharmacy and Pharmacology Communications 1998, 4, 241-243, Rosseels, G. et al. Eur. J. Med. Chem., 1975, 10, 579-584 and Casagrande, C. et al. Farmaco, 1971, 26, 1059-1073 refer to indolizine compounds having anti-inflammatory activity. In none of these publications is it suggested that the disclosed indolizine compounds may have CRTH2 activity or be useful in the treatment of PGD2 mediated diseases or conditions.

### DETAILED DESCRIPTION OF THE INVENTION

According to the present invention, there is provided a compound of formula (I) or a salt, N-oxide, hydrate or solvate thereof:

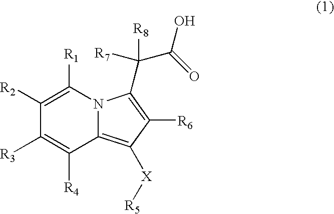

wherein
R₁, R₂. R₃ and R₄ each independently are hydrogen, C₁-C₆alkyl, fully or partially fluorinated C₁-C₆alkyl, halo, —S(O)ₙR₁₀, —SO₂N(R₁₀)₂, —N(R₁₀)₂, —C(O)N(R₁₀)₂, —NR₁₀C(O)R₉, —CO₂R₁₀, —C(O)R₉, —NO₂, —CN or —OR₁₁;
wherein each R₉ is independently C₁-C₆alkyl, aryl, heteroaryl;
R₁₀ is independently hydrogen, C₁-C₆alkyl, aryl, or heteroaryl;
R₁₁ is hydrogen, C₁-C₆alkyl, fully or partially fluorinated C₁-C₆alkyl or a group —SO₂R₁₀;
n is 0, 1 or 2;
   R₅ is C₁-C₆alkyl, fully or partially fluorinated C₁-C₆alkyl, C₁-C₆alkenyl, C₁-C₆alkynyl, optionally substituted aryl, or optionally substituted heteroaryl;
   R₆ is hydrogen, C₁-C₆alkyl or fully or partially fluorinated C₁-C₆alkyl;
   R₇ and R₈ are independently hydrogen or C₁-C₆alkyl, or R₇ and R₈ together with the atom to which they are attached form a cycloalkyl group; and
   X is —CHR₆—, —S(O)ₙ—, —C(O)—, —NR₆SO₂— or —SO₂NR₆— wherein n is 0, 1 or 2.

For the avoidance of doubt the numbering system used for the indolizine ring system is shown below:

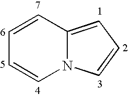

Compounds (I) with which the invention is concerned are CRTH2 receptor antagonists, but they may also have beneficial effects at other prostanoid receptors, such as the DP receptor or the thromboxane A₂ receptor. The publication referred to in the preceding paragraph is concerned with indolizine analogues of indomethacin, and does suggest that any of the disclosed compounds have the foregoing activities of the compounds of this invention.

A second aspect of the invention is a pharmaceutical composition comprising a compound of formula (I), or a salt, N-oxide, hydrate or solvate thereof, in admixture with a pharmaceutically acceptable carrier or excipient.

A third aspect of the invention is a compound of formula (I), or a salt, N-oxide, hydrate or solvate thereof, for use in therapy.

A fourth aspect of the invention is the use of a compound of formula (I), or a salt, N-oxide, hydrate or solvate thereof, in the manufacture of a medicament for the treatment of a disease in which a CRTH2 antagonist can prevent, inhibit or ameliorate the pathology and/or symptomotology of the disease.

A fifth aspect of the invention is a method for treating a disease in a patient in which a CRTH2 antagonist can prevent, inhibit or ameliorate the pathology and/or symptomotology of the disease, which method comprises administering to the patient a therapeutically effective amount of compound of formula (I), or a salt, N-oxide, hydrate or solvate thereof.

### DESCRIPTION OF THE INVENTION

In particular, compounds with which the invention is concerned are useful in the treatment of disease associated with elevated levels of prostaglandin D2 (PGD2) or one or more active metabolites thereof.

Examples of such diseases include asthma, rhinitis, allergic airway syndrome, allergic rhinobronchitis, bronchitis, chronic obstructive pulmonary disease (COPD), nasal polyposis, sarcoidosis, farmer's lung, fibroid lung, cystic fibrosis, chronic cough, conjunctivitis, atopic dermatitis, Alzheimer's disease, amyotrophic lateral sclerosis, AIDS dementia complex, Huntington's disease, frontotemporal dementia, Lewy body dementia, vascular dementia, Guillain-Barre syndrome, chronic demyelinating polyradiculoneurophathy, multifocal motor neuropathy, plexopathy, multiple sclerosis, encephalomyelitis, panencephalitis, cerebellar degeneration and encephalomyelitis, CNS trauma, migraine, stroke, rheumatoid arthritis, ankylosing spondylitis, Behcet's Disease, bursitis, carpal tunnel syndrome, inflammatory bowel disease, Crohn's disease, ulcerative colitis, dermatomyositis, Ehlers-Danlos Syndrome (EDS), fibromyalgia, myofascial pain, osteoarthritis (OA), osteonecrosis, psoriatic arthritis, Reiter's syndrome (reactive arthritis), sarcoidosis, scleroderma, Sjogren's Syndrome, soft tissue disease, Still's Disease, tendinitis, polyarteritis Nodossa, Wegener's Granulomatosis, myositis (polymyositis dermatomyositis), gout, atherosclerosis, lupus erythematosus, systemic lupus erythematosus (SLE), type I diabetes, nephritic syndrome, glomerulonephritis, acute and chronic renal failure, eosinophilia fascitis, hyper IgE syndrome, sepsis, septic shock, ischemic reperfusion injury in the heart, allograft rejection after transplantations, and graft versus host disease.

However, the compounds with which the invention is concerned are primarily of value for the treatment of asthma, chronic obstructive pulmonary disease, rhinitis, allergic airway syndrome, and allergic rhinobronchitis.

### Terminology

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein, the term “fully or partially fluorinated Cₐ-C_{b}alkyl” wherein a and b are integers refers to a straight or branched chain alkyl radical having from a to b carbon atoms in which the hydrogen atoms all replaced by fluorine (fully fluorinated) or in which some of the hydrogen atoms are replaced by fluorine (partially fluorinated). The term includes, for example —CF₃, —CHF₂, —CFH₂, and CF₃CH₂—.

As used herein the term “(Cₐ-C_{b})alkenyl” wherein a and b are integers refers to a straight or branched chain alkenyl moiety having from a to b carbon atoms having at least one double bond of either E or Z stereochemistry where applicable. The term includes, for example, vinyl, allyl, 1- and 2-butenyl and 2-methyl-2-propenyl.

As used herein the term “Cₐ-C_{b} alkynyl” wherein a and b are integers refers to straight chain or branched chain hydrocarbon groups having from two to six carbon atoms and having in addition one triple bond. This term would include for example, ethynyl, 1- and 2-propynyl, 1-, 2- and 3-butynyl, 1, 2-, 3- and 4-pentynyl, 1-, 2-, 3-, 4- and 5-hexynyl, 3-methyl-1-butynyl, and 1-methyl-2-pentynyl.

As used herein the term “carbocyclic” refers to a mono-, bi- or tricyclic radical having up to 16 ring atoms, all of which are carbon, and includes aryl and cycloalkyl.

As used herein the term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzothienyl, furyl, benzofuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyridazinyl, triazinyl, indolyl and indazolyl.

As used herein the unqualified term “heterocyclyl” or “heterocyclic” includes “heteroaryl” as defined above, and in addition means a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, quinolyl, morpholinyl, benzofuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, cycloalkyl, (C₁-C₆)alkoxy, hydroxy, hydroxy(C₁-C₆)alkyl, mercapto, mercapto(C₁-C₆)alkyl, (C₁-C₆)alkylthio, phenyl, monocyclic heteroaryl having 5 or 6 ring atoms, halo (including fluoro, bromo and chloro), trifluoromethyl, trifluoromethoxy, nitro; nitrile (—CN), oxo, —COOH, —COOR^{A}, —COR^{A}, —SO₂R^{A}, —CONH₂, —SO₂NH₂, —CONHR^{A}, —SO₂NHR_{A}, —CONR^{A}R^{B}, —SO₂NR^{A}R^{B}, —NH₂, —NHR^{A}, —NR^{A}R^{B}, —OCONH₂, —OCONHR^{A}, OCONR^{A}R^{B}, —NHCOR^{A}, —NHCOOR^{A}, NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl, or monocyclic heterocyclic group having 5 or 6 ring atoms (examples of the latter being morpholinyl, piperidinyl, piperizinyl, 4-methylpiperidinyl, and tetrahydropyrrolyl). An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Specific salts with bases include the benzathine, calcium, diolamine, meglumine, olamine, potassium, procaine, sodium, tromethamine and zinc salts. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesulfonic, glutamic, lactic, and mandelic acids and the like.

Compounds with which the invention is concerned which may exist in one or more stereoisomeric form, because of the presence of asymmetric atoms or rotational restrictions, can exist as a number of stereoisomers with R or S stereochemistry at each chiral centre or as atropisomers with R or S stereochemistry at each chiral axis. The invention includes all such enantiomers and diastereoisomers and mixtures thereof.

Use of prodrugs, such as esters, of compounds (I) with which the invention is concerned is also part of the invention. “Prodrug” means a compound which is convertible in vivo by metabolic means (e.g. by hydrolysis, reduction or oxidation) to a compound of formula (I). For example an ester prodrug of a compound of formula (I) may be convertible by hydrolysis in vivo to the parent molecule. Suitable esters of compounds of formula (I) are for example acetates, citrates, lactates, tartrates, malonates, oxalates, salicylates, propionates, succinates, fumarates, maleates, methylene-bis-β-hydroxynaphthoates, gentisates, isethionates, di-p-toluoyltartrates, methanesulphonates, ethanesulphonates, benzenesulphonates, p-toluene-sulphonates, cyclohexylsulphamates and quinates. Examples of ester prodrugs are those described by F. J. Leinweber, Drug Metab. Res., 1987, 18, 379. As used in herein, references to the compounds of formula (I) are meant to also include the prodrug forms.

### The Variables R₁-R₈

For use in accordance with the invention, the following structural characteristics are currently preferred, in any compatible combination, in the compounds (I):
R₁, R₂, R₃ and R₄ are preferably electron-deficient or electron-withdrawing substituents, and may be independently selected from, for example, hydrogen methyl, ethyl, trifluoromethyl, fluoro, chloro, bromo, —NO₂, —CN, —SO₂R₉, —SO₂N(R₁₀)₂, —C(O)N(R₁₀)₂, —NR₁₀C(O)R₉, —CO₂R₁₀, and —C(O)R₉, wherein R₉, R₁₀ and R₁₁ are as defined above with reference to formula (I). Thus: R₉ may be selected from, for example, methyl, ethyl, and phenyl; R₁₀ may be selected from, for example, hydrogen, methyl, ethyl, and phenyl; and R₁₁ may be selected from, for example, methyl, trifluoromethyl, ethyl, phenyl, —SO₂H and —SO₂CH₃. Currently preferred are compounds in which R₁, R₂, R₃ and R₄ are independently selected from hydrogen, chloro, fluoro, cyano, methyl, trifluoromethyl. Often, two of R₁, R₂, R₃ and R₄ be hydrogen, while the others, for example R₂ and R₃, are independently selected from hydrogen, chloro, fluoro, cyano, methyl, and trifluoromethyl.
R₅ may be, for example, methyl, ethyl, n- or iso-propyl, trifluoromethyl, allyl, optionally substituted phenyl or naphthyl; or optionally substituted monocyclic heteroaryl having 5 or 6 ring atoms, such as optionally substituted pyridyl, pyrimidinyl, furyl, thienyl, imidazolyl, oxazolyl or isoxazolyl or pyrrolyl; or optionally substituted bicyclic heteroaryl having 8 to 10 ring atoms, such as quinolinyl, benzothiazolyl, indolyl, and benzimidazolyl. Optional substituents in any such ring systems may be any of those listed above with reference to the term “substituted”, for example chloro, fluoro, trifluoromethyl, methylsulfonyl, ethylsulfonyl, carbamate, methylcarbamate, methylaminosulfonyl, ethylaminosulfonyl, methylsulfonylamino, ethylsulfonylamino, morpholin-1-ylsulfonyl, piperidin-1-ylsulfonyl, piperazin-1-ylsulfonyl, 4-methylpiperazin-1-ylsulfonyl, and tetrahydropyrrole-1-ylsulfonyl.
The divalent radical —X— may be, for example, —CH₂—, —CH(C₁-C₃ alkyl)-S—, —S(O)—, —SO₂—, —C(O)—, —NHSO₂— or —SO₂NH—. Currently it is preferred that —X— be —S—, —SO₂—, —CH₂—, or —C(O)—.
R₆ may be, for example, hydrogen, methyl, ethyl or trifluoromethyl. Currently it is preferred that R₆ be methyl.
R₇ and R₈ may both be hydrogen, or one of R₇ and R₈ may be, for example, methyl while the other is hydrogen, or R₇ and R₈ taken together with the carbon atom to which they are attached may form, for example, a cyclopropyl, cyclopentyl or cyclohexyl ring. Presently it is preferred that both R₇ and R₈ be hydrogen.

The carboxylic acid group attached to —C(R₇)(R₈)— may be esterified as an ester which is hydrolysed in vivo to release the carboxylic acid, and in such cases the compound is an ester prodrug of compounds (I).

In one particular subclass of the compounds with which the invention is concerned, R₁, R₂, R₃ and R₄ are independently selected from hydrogen, chloro, fluoro, cyano, methyl, and trifluoromethyl; X is —S—, —SO₂—; —CH₂— or —C(O)—, R₅ is optionally substituted phenyl or optionally substituted heteroaryl such as optionally substituted quinolinyl or benzothiazolyl, R₆ is methyl, and R₇ and R₈ are hydrogen. In this subclass, when R₅ is optionally substituted phenyl or heteroaryl, one or two substituents may be present, independently selected from chloro, fluoro, methylsulfonyl, ethylsulfonyl, carbamate, methylcarbamate, methylaminosulfonyl, ethylaminosulfonyl, methylsulfonylamino, ethylsulfonylamino, morpholin-1-ylsulfonyl, piperidin-1-ylsulfonyl, piperazin-1-ylsulfonyl, 4-methylpiperazin-1-ylsulfonyl, and tetrahydropyrrole-1 ylsulfonyl.

Specific examples of compounds with which the invention is concerned include those of the examples herein.

### Compositions

As mentioned above, the compounds with which the invention is concerned are CRTH2 receptor antagonists, and are useful in the treatment of diseases which benefit from such modulation. Examples of such diseases are referred to above, and include asthma, rhinitis, allergic airway syndrome, and allergic rhinobronchitis.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial, as is required in the pharmaceutical art. In general, the daily dose range will lie within the range of from about 0.001 mg to about 100 mg per kg body weight of a mammal, often 0.01 mg to about 50 mg per kg, for example 0.1 to 10 mg per kg, in single or divided doses. On the other hand, it may be necessary to use dosages outside these limits in some cases.

The compounds with which the invention is concerned may be prepared for administration by any route consistent with their pharmacokinetic properties. Orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the drug may be made up into a cream, lotion or ointment. Cream or ointment formulations which may be used for the drug are conventional formulations well known in the art, for example as described in standard textbooks of pharmaceutics such as the British Pharmacopoeia.

The drug may also be formulated for inhalation, for example as a nasal spray, or dry powder or aerosol inhalers. For delivery by inhalation, the active compound is preferably in the form of microparticles. They may be prepared by a variety of techniques, including spray-drying, freeze-drying and micronization. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems.

The active ingredient may also be administered parenterally in a sterile medium. Depending on the vehicle and concentration used, the drug can either be suspended or dissolved in the vehicle. Advantageously, adjuvants such as a local anaesthetic, preservative and buffering agents can be dissolved in the vehicle.

Other compounds may be combined with compounds of this invention of formula [I] for the prevention and treatment of prostaglandin-mediated diseases. Thus the present invention is also concerned with pharmaceutical compositions for preventing and treating PGD₂-mediated diseases comprising a therapeutically effective amount of a compound of the invention of formula [I] and one or more other therapeutic agents. Suitable therapeutic agents for a combination therapy with compounds of formula [1] include, but are not limited to: (1) corticosteroids, such as fluticasone, ciclesonide or budesonide; (2) β2-adrenoreceptor agonists, such as salmeterol, indacaterol or formoterol; (3) leukotriene modulators, for example leukotriene antagonists such as montelukast, zafirulast or praniukast or leukotriene biosynthesis inhibitors such as Zileuton or BAY-1005; (4) anticholinergic agents, for example muscarinic-3 (M3) receptor antagonists such as tiotropium bromide; (5) phosphodiesterase-IV (PDE-IV) inhibitors, such as roflumilast or cilomilast; (6) antihistamines, for example selective histamine-1 (H1) receptor antagonists, such as fexofenadine, citirizine, loratidine or astemizole; (7) antitussive agents, such as codeine or dextramorphan; (8) non-selective COX-1/COX-2 inhibitors, such as ibuprofen or ketoprofen; (9) COX-2 inhibitors, such as celecoxib and rofecoxib; (10) VLA-4 antagonists, such as those described in WO97/03094 and WO97/02289; (11) TACE inhibitors and TNF-α inhibitors, for example anti-TNF monoclonal antibodies, such as Remicade and CDP-870 and TNF receptor immunoglobulin molecules, such as Enbrel; (12) inhibitors of matrix metalloprotease, for example MMP12; (13) human neutrophil elastase inhibitors, such as those described in WO2005/026124, WO2003/053930 and WO06/082412; (14) A2a agonists such as those described in EP1052264 and EP1241176 (15) A2b antagonists such as those described in WO2002/42298; (16) modulators of chemokine receptor function, for example antagonists of CCR3 and CCR8; (17) compounds which modulate the action of other prostanoid receptors, for example a DP receptor antagonist or a thromboxane A₂ antagonist; and (18) agents that modulate Th2 function, such as PPAR agonists

The weight ratio of the compound of the invention to the second active ingredient may be varied and will depend upon the effective dose of each ingredient. Generally, an effective dose of each will be used.

### Methods of Synthesis

The present invention is also concerned with processes for preparing the compounds of this invention.

The compounds of formula (I) of the present invention can be prepared according to the procedures of the following schemes and examples, using appropriate materials, and are further exemplified by the following specific examples. Moreover, by utilizing the procedures described with the disclosure contained herein, one of ordinary skill in the art can readily prepare additional compounds of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention of formula (I) may be isolated in the form of their pharmaceutically acceptable salts, such as those described previously herein above. The free acid form corresponding to isolated salts can be generated by acidification with a suitable acid such as acetic acid and hydrochloric acid and extraction of the liberated free acid into an organic solvent followed by evaporation. The free acid form isolated in this manner can be further converted into another pharmaceutically acceptable salt by dissolution in an organic solvent followed by addition of the appropriate base and subsequent evaporation, precipitation, or crystallisation.

It may be necessary to protect reactive functional groups (e.g. hydroxy, amino, thio or carboxy) in intermediates used in the preparation of compounds of formula (I) to avoid their unwanted participation in a reaction leading to the formation of compounds of formula (I). Conventional protecting groups, for example those described by T. W. Greene and P. G. M. Wuts in “Protective groups in organic chemistry” John Wiley and Sons, 1999, may be used.

Compounds of formula (I) wherein X is a —SO₂— group (sulfone) are represented by compounds of formula (I-a). Compounds of formula (I-a) wherein R^{a} is as defined for R⁵ and R^{b} is as defined for R⁶ in formula (I) may be prepared according to the route outlined in Scheme 1.

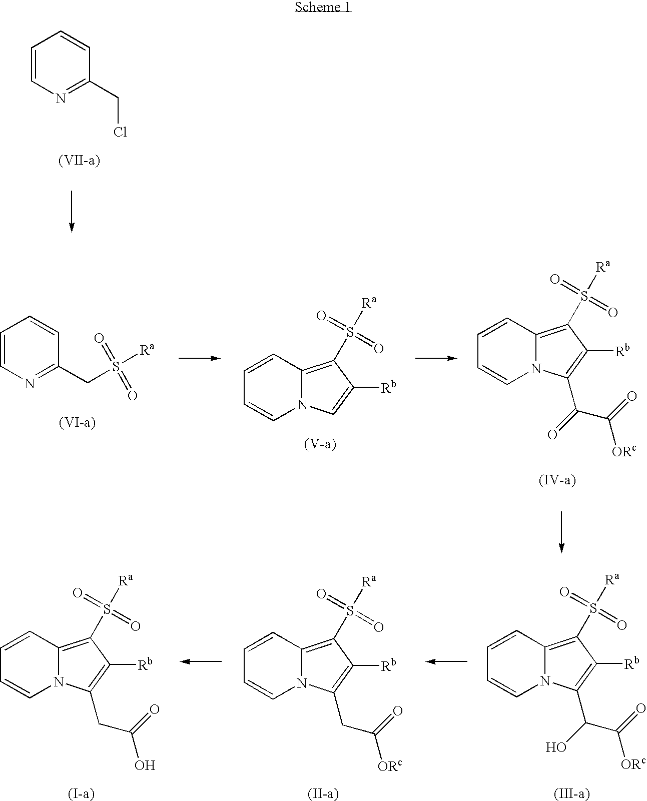

Compounds of formula (I-a) may be prepared from compounds of formula (II-a) wherein R^{a} and R^{b} are as defined above and R^{c} is a lower alkyl group, by hydrolysis of the ester group under standard conditions familiar to those skilled in the art. For example, by treatment with a metal hydroxide such as lithium hydroxide in a polar protic solvent such as an alcohol, preferably methanol, in the presence of water. The reaction may be conducted at a temperature between 0° C. and the reflux temperature of the solvent, preferably at ambient temperature.

Compounds of formula (II-a) may be prepared from compounds of formula (III-a) wherein R^{a}, R^{b} and R^{c} are as defined above, by reduction (dihydroxylation) with triphenylphosphine and iodine in an inert solvent, such as toluene, at a temperature between ambient temperature and the reflux temperature of the solvent, preferably at 70-90° C.

Compounds of formula (III-a) may be prepared from compounds of formula (IV-a) wherein R^{a}, R^{b} and R^{c} are as defined above, by treatment with a reducing agent, such as a borohydride, particularly sodium borohydride, in a polar solvent, such as an alcohol/water mixture, for example methanol/water. The reaction is carried out at a temperature below 20° C., preferably at −40° C.

Compounds of formula (IV-a) may be prepared from compounds of formula (V-a) wherein R^{a} and R^{b} are as defined above, by treatment with a compound of formula (VIII);

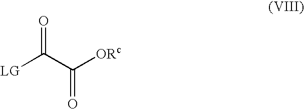

wherein R^{c} is as defined above and LG represents a leaving group, particularly a halogen. When LG is chlorine and R^{c} is methyl, formula (VIII) represents commercially available methyl chlorooxoacetate. The reaction is conducted in an aprotic solvent, such as tetrahydrofuran at a temperature between 0 and 50° C., preferably 20° C. Compounds of formula (VIII) are commercially available or prepared by known methods.

Compounds of formula (V-a) may be prepared from compounds of formula (VI-a) wherein R^{a} is as defined above, by treatment with a compound of formula (IX);

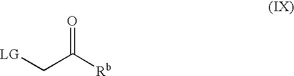

wherein R^{b} is as defined above. The reaction takes place in the presence of a suitable base, for example a metal carbonate such as potassium carbonate in a suitable solvent such as acetone or methyl ethyl ketone, at a temperature between 20° C. and the reflux temperature of the solvent, preferably the reflux temperature. Compounds of formula (IX) are known or may be prepared from known compounds according to methods known to those skilled in the art.

Compounds of formula (VI-a) may be prepared from compounds of formula (VII-a) by treatment with a compound of formula (X);

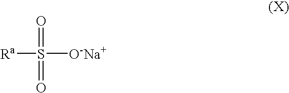

wherein R^{a} is as defined above. The reaction is carried out in a polar solvent such as butanol in the presence of a base such as sodium acetate, at a temperature between 20° C. and the reflux temperature of the solvent, preferably between and 50 and 80° C. Compounds of formula (VII-a) and (X) are known in the literature or prepared from known compounds by well known methods.

Compounds of formula (I) where X is a —S— group (sulfide) are represented by formula (I-b). Compounds of formula (I-b) wherein R^{a} and R^{b} are as defined for R⁵ and R⁶ in formula (I) above, may be prepared according to the routes outlined in Scheme 2.

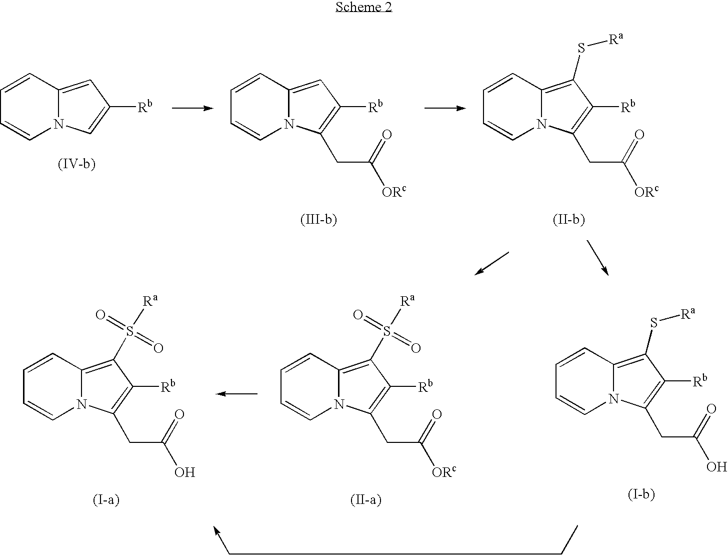

Compounds of formula (I-b) may be prepared from compounds of formula (II-b) wherein R^{a} and R^{b} are as described above and R^{c} is an alkyl group, using methods described above for the preparation of compounds of formula (I-a) from compounds of formula (II-a) (Scheme 1). Compounds of formula (II-b) may be prepared from compounds of formula (III-b) wherein R^{b} is as defined above and R^{c} is an alkyl group by treatment with a disulfide of formula (XI);

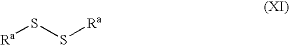

The reaction is carried out in a polar protic solvent, particularly an alcohol such as ethanol, at the reflux temperature of the solvent. Compounds of formula (XI) are commercially available or are known compounds or can readily be prepared from known compounds using methods described in the literature. Alternatively a sulfenyl halide may be used in place of the disulfide [XI].

Compounds of formula (III-b) may be prepared from compounds of formula (IV-b) wherein R^{b} is as described above by reaction with a compound of formula (XII);

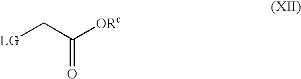

wherein R^{c} is as described above and LG is a leaving group, such as a halogen, particularly chlorine, bromine or an iodine atom. The reaction is conducted in a non-protic solvent such as dioxane in the presence of a Friedel-Crafts catalyst such as aluminium trichloride at a temperature between ambient temperature and the reflux temperature of the solvent, preferably 20° C.

Alternatively, compounds of formula (III-b) may be prepared from compounds of formula (IV-b) wherein R^{b} is as described above by reaction with a compound of formula (XIII), in the presence of a suitable catalyst, such as copper bronze or copper powder. Compounds of general formula (XIII) are commercially available or can be prepared by methods well known to those skilled in the art.

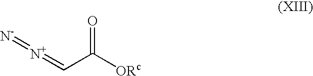

Compounds of formula (IV-b) are known in the art, for example, Holland et al, J. Chem. Soc. 1955, 1657-1660.

Using the route outlined in Scheme 2, compounds of formula (I-a) may be prepared from compounds of formula (II-b) by oxidation/ester hydrolysis via compounds of formula (II-a) or (1-b).

Compounds of formula (I) wherein X is a —C(O)— group (carbonyl) are represented by formula (I-c). Compounds of formula (I-c) wherein R^{a} and R^{b} are as defined for R⁵ and R⁶ above may be prepared according to the route outlined in Scheme 3.

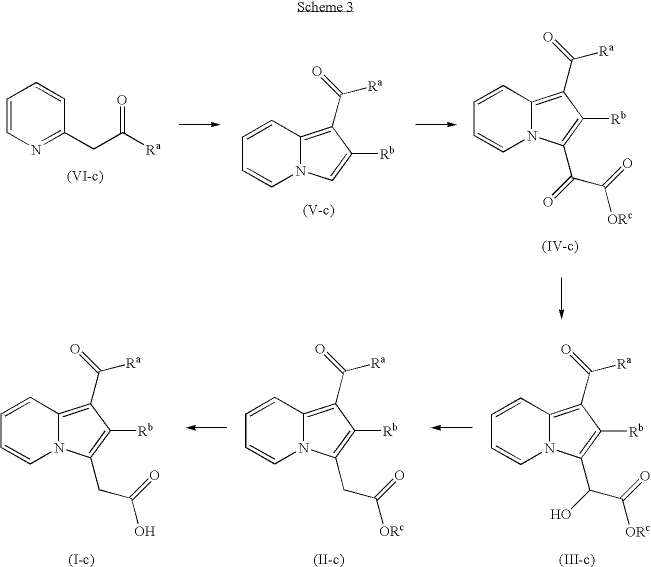

Compounds of formula (I-c) may be prepared over several steps from compounds of formula (VI-c) using methods described above (Scheme 1) for the preparation of compounds of formula (I-a) from compounds of formula (VI-a), wherein R^{a}, R^{b} and R^{c} are as defined above.

Compounds of formula (VI-c) are known compounds or may be prepared from known compounds by methods familiar to one skilled in the art.

Compounds of formula (I) wherein X is a —CH₂— group are represented by formula (I-d). Compounds of formula (I-d) wherein R^{a} and R^{b} are as defined for R⁵ and R⁶ above may be prepared according to the route outlined in Scheme 4.

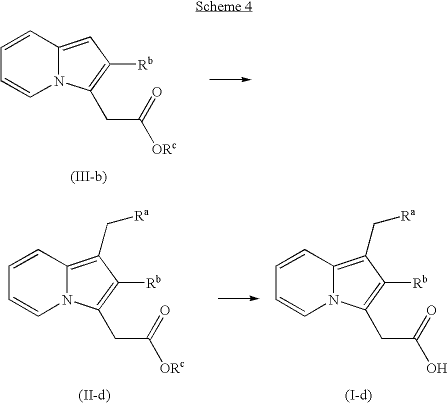

Compounds of formula (I-d) may be prepared from compounds of formula (II-d) wherein R^{a}, R^{b} and R^{c} are as described above, using methods described above for the preparation of compounds of formula (I-a) from compounds of formula (II-a) (Scheme 1). Compounds of formula (II-d) may be conveniently prepared by reaction with a compound of general formula (XIV), wherein R^{a} is defined as above;

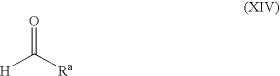

under acidic reductive conditions, for example a mixture of trifluoroacetic acid and triethylsilane. Compounds of general formula (XIV) are commercially available or can be prepared by methods well known to those skilled in the art.

### Biological Methods

Compounds of the invention can be tested using the following biological test methods to determine their ability to displace PGD₂ from the CRTH2 receptor and for their ability to antagonize the functional effects of PGD₂ at the CRTH2 receptor in a whole cell system.

### Radioligand Binding Assay

The receptor binding assay is performed in a final volume of 200 μL binding buffer [10 mM BES (pH 7.4), 1 mM EDTA, 10 mM manganese chloride, 0.01% BSA] and 1 nM [³H]-PGD₂ (Amersham Biosciences UK Ltd). Ligands are added in assay buffer containing a constant amount of DMSO (1% by volume). Total binding is determined using 1% by volume of DMSO in assay buffer and non-specific binding is determined using 10 μM of unlabeled PGD₂ (Sigma). Human embryonic kidney (HEK) cell membranes (3.5 μg) expressing the CRTH2 receptor are incubated with 1.5 mg wheatgerm agglutinin SPA beads and 1 nM [³H]-PGD₂ (Amersham Biosciences UK Ltd) and the mixture incubated for 3 hours at room temperature. Bound [³H]-PGD₂ is detected using a Microbeta TRILUX liquid scintillation counter (Perkin Elmer). Compound IC₅₀ value is determined using a 6-point dose response curve in duplicate with a semi-log compound dilution series. IC₅₀ calculations are performed using Excel and XLfit (Microsoft), and this value is used to determine a Ki value for the test compound using the Cheng-Prusoff equation.

### Functional Assays

### GTPγS Assay

The GTPγS Assay is performed in a final volume of 200 mL assay buffer (20 mM HEPES pH 7.4, 10 mM MgCl₂, 100 mM NaCl, 10 μg/mL saponin). DMSO concentrations are kept constant at 1% by volume. Human embryonic kidney (HEK) cell membranes (3.5 μg) expressing the CRTH2 receptor are incubated with the compounds for 15 min at 30° C. prior to addition of PGD₂ (30 nM final concentration) and GTP (10 μM final concentration). The assay solutions are then incubated for 30 minutes at 30° C., followed by addition of [³⁵S]-GTPγS (0.1 nM final concentration). The assay plate is than shaken and incubated for 5 minutes at 30° C. Finally, SPA beads (Amersham Biosciences, UK) are added to a final concentration of 1.5 mg/well and the plate shaken and incubated for 30 minute at 30° C. The sealed plate is centrifuged at 1000 g for 10 mins at 30° C. and the bound [³⁵S]-GTPγS is detected on Microbeta scintillation counter (Perkin Elmer). Compound IC₅₀ value is determined using a 6-point dose response curve in duplicate with a semi-log compound dilution series. IC₅₀ calculations are performed using Excel and XLfit (Microsoft), and this value is used to determine a Ki value for the test compound using the Cheng-Prusoff equation.

### Calcium Mobilization Assay

Stable CHO-K1 cells co-expressing the CRTH2 receptor and the G-protein Gα16 are seeded (40,000 cells per well in a plating volume of 75 μL in F-12 Hams supplemented with 1% foetal bovine serum) into collagen-coated 96-well plates 24 hours prior to the assay. The cells are then loaded with a fluorescence-imaging plate reader (FLIPR) calcium kit dye (Calcium 3 kit, Molecular Devices Ltd) containing 5 mM final concentration of probenecid and incubated at 37° C. for 1 hour in a 5% CO₂ atmosphere. The fluorescence emission caused by intracellular calcium mobilization elicited by the PGD₂ at the CRTH2 receptor is determined with a FLEXstation benchtop scanning and integrated fluid transfer workstation (Molecular Devices Ltd). To detect antagonists and determine compound IC₅₀, compounds are pre-incubated at varying concentrations with the loaded cells for 15 minutes at 37° C., 5% CO₂, prior to the addition of the agonist at its EC₈₀ value. Compounds and agonist are added in Hanks balanced salt solution containing 20 mM HEPES and 0.1% BSA). The fractional response values for each well are calculated by subtracting the basal response from the peak response. Results are calculated as the mean of triplicate wells using Excel and XLfit (Microsoft).

### Eosinophil Chemotaxis Assay

Eosinophils chemotaxis assay is performed immediately after isolating eosinophils from freshly collected blood. Whole citrated blood is spun in Accuspin tubes (Sigma, as are all other reagents) containing 15 mL histopaque at 800 g for 15 min (brake off). The granulocyte containing pellet is re-suspended in 20 ml of 6% dextrane, than topped up to 50 mL with PBS. The tubes are inverted 3-4 times and left to sediment for 45 minutes. The supernatant is collected and centrifuged at 800 g for 10 minutes. The resulting pellet is re-suspended in 2 mL PBS followed by 24 mL sterile water. The remaining read blood cells are than lysed by adding 8 mL 3.5% NaCl and the suspension centrifuged at 800 g for 10 minutes. The pellet is re-suspended in 1 mL of running buffer (PBS with 0.5% BSA and 2 mM EDTA) and the granulocytes counted in a haemocytometer. The cell suspension is centrifuged again at 800 g for 10 mins and the granulocyte pellet resuspended in 50 μL buffer per 50 million cells. An equal volume of Miltenyi CD16 beads is added (Miltenyi Biotec, Germany) and the beads are incubated with cells for 30 mins at 4° C. Wash the granulocyte/bead suspension with 1-2 mL buffer per 10 million cells, spin down and re-suspend in 500□L buffer. Load the cell/bead suspension onto the Miltenyi column. Allow the cells to pass into the column and then elute the unlabelled (negative) fraction (eosinophils) with 30 mL buffer.

The chemotaxis assay is performed in disposable 96-well chambers (Neuoroprobe) The chemoattractant and control solutions are placed into appropriate wells of the lower section of the chamber (29 μL, in PBS with 0.5% BSA and 2 mM EDTA) and the framed membrane is fixed into place. 25 μL of the cell suspension is pipetted onto the membrane above each of the filled lower wells (200,000 cells per 25 uL) and the chambers incubated for 2 hrs at 37° C. After the incubation, the remaining cells are aspirated from the top of membrane and the plate centrifuged at 200 rpm for 10 min. The membrane is than removed, the supernatants aspirated from each well and the plate frozen for at least 30 mins at −80° C. Upon thawing 20 μL of prepared Cyquant reagent is added to each well (Cyquant cell proliferation kit, Invitrogen). The fluorescence is measured using a plate reader with appropriate settings (Victor V, Perkin Elmer).

### EXAMPLES

The invention will now be described with reference to the following examples.

¹H NMR spectra were recorded at ambient temperature using a Varian Unity Inova (400 MHz) spectrometer with a triple resonance 5 mm probe spectrometer. Chemical shifts are expressed in ppm relative to tetramethylsilane. The following abbreviations have been used: br s=broad singlet, s=singlet, d=doublet, dd=double doublet, t=triplet, q=quartet, m=multiplet.

Mass Spectrometry (LCMS) experiments to determine retention times and associated mass ions were performed using the following methods:

Method A: experiments were performed on a Micromass Platform LCT spectrometer with positive ion electrospray and single wavelength UV 254 nm detection using a Higgins Clipeus C18 5 μm 100×3.0 mm column and a 2 mL/minute flow rate. The initial solvent system was 95% water containing 0.1% formic acid (solvent A) and 5% acetonitrile containing 0.1% formic acid (solvent B) for the first minute followed by a gradient up to 5% solvent A and 95% solvent B over the next 14 minutes. The final solvent system was held constant for a further 2 minutes.

Method B: experiments were performed on a Micromass Platform LC spectrometer with positive and negative ion electrospray and ELS/Diode array detection using a Phenomenex Luna C18(2) 30×4.6 mm column and a 2 mL/minute flow rate. The solvent system was 95% solvent A and 5% solvent B for the first 0.50 minutes followed by a gradient up to 5% solvent A and 95% solvent B over the next 4 minutes. The final solvent system was held constant for a further 0.50 minutes

Microwave experiments were carried out using a Personal Chemistry Smith Synthesizer™, which uses a single-mode resonator and dynamic field tuning, both of which give reproducibility and control. Temperatures from 40-250° C. can be achieved, and pressures of up to 20 bar can be reached. Two types of vial are available for this processor, 0.5-2.0 mL and 2.0-5.0 mL.

Reverse-phase preparative HPLC purifications were carried out using Genesis 7 micron C-18 bonded silica stationary phase in columns 10 cm in length and 2 cm internal diameter. The mobile phase used was mixtures of acetonitrile and water (both buffered with 0.1% v/v trifluoroacetic acid) with a flow rate of 10 mL per minute and typical gradients of 40 to 90% organic modifier ramped up over 30 to 40 minutes. Fractions containing the required product (identified by LC-MS analysis) were pooled, the organic fraction removed by evaporation, and the remaining aqueous fraction lyophilised, to give the final product.

### Example 1

### [1-(4-methanesulfonylphenylsulfanyl)-2-methylindolizin-3-yl]acetic acid

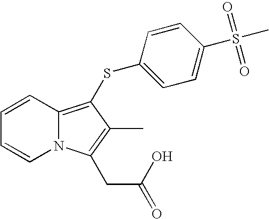

### Preparation 1a

### 2-methylindolizine

A mixture of 1-chloropropan-2-one (32 mL), 2-methylpyridine (40 mL) and acetone (40 mL) was heated at reflux for 2 hours. The mixture was cooled to room temperature and the resulting precipitate collected by filtration and washed with dichloromethane. The precipitate was dissolved in water (480 mL), treated with sodium hydrogen carbonate (36 g) and distilled under atmospheric pressure. The distillate was extracted with diethyl ether and the combined extracts washed with saturated aqueous sodium chloride solution and dried over magnesium sulfate. The solvent was removed under reduced pressure to afford title compound as a yellow/brown solid, 7.6 g.

¹H NMR (CDCl₃): δ 2.30 (s, 3H), 6.25 (s, 1H), 6.35 (t, J=6.7 Hz, 1H), 6.60 (t, J=7.6 Hz, 1H), 7.10 (s, 1H), 7.25 (d, J=6.7 Hz, 1H), 7.80 (d, J=6.7 Hz, 1H).

### Preparation 2b

### (2-methylindolizin-3-yl)acetic acid ethyl ester

A mixture of 2-methylindolizine (14 g), iodoacetic acid ethyl ester (15 mL), aluminium trichloride (0.69 g) and 1,4-dioxane (100 mL) was stirred at room temperature for 5 days. The mixture was diluted with water, extracted with dichloromethane and the combined extracts dried over magnesium sulfate. The solvent was removed under reduced pressure and the residue purified by column chromatography on silica gel, eluting with a mixture of cyclohexane and dichloromethane (9:1 to 6:1 by volume) to afford title compound as a yellow solid, 8.5 g.

¹H NMR (CDCl₃): δ 1.20 (t, J=7.1 Hz, 3H), 2.30 (s, 3H), 3.85 (s, 2H), 4.10 (q, J=7.1 Hz, 2H), 6.30 (br s, 1H), 6.50 (t, J=6.1 Hz, 1H), 6.65 (m, 1H), 7.30 (d, J=8.8 Hz, 1H), 7.85 (m, 1H).

MS: ESI (+ve) (Method B): 218 (M+H)⁺, Retention time 3.8 min.

### Preparation 1c

### [1-(4-methanesulfonylphenylsulfanyl)-2-methylindolizin-3-yl]acetic acid ethyl ester

A mixture of (2-methylindolizin-3-yl)acetic acid ethyl ester (0.17 g), bis[4-(methylsulfonyl)phenyl]disulfide (1.16 g), iodine (one crystal) and ethanol (5.0 mL) was heated at reflux for 16 hours. The solvent was removed under reduced pressure and the residue purified by column chromatography on silica gel, eluting with a mixture of dichloromethane and ethyl acetate to afford title compound, 0.2 g.

MS: ESI (+ve) (Method B): 404 (M+H)⁺, Retention time 3.9 min.

### Preparation 1d

### [1-(4-methanesulfonylphenylsulfanyl)-2-methylindolizin-3-yl]acetic acid

A solution of [1-(4-methanesulfonylphenylsulfanyl)-2-methylindolizin-3-yl]acetic acid ethyl ester (0.20 g), methanol (1.5 mL), lithium hydroxide (0.08 g) and water (0.5 mL) was stirred at room temperature for 90 minutes. The mixture was diluted with 1.0 M aqueous hydrochloric acid and extracted with dichloromethane. The combined extracts were dried over magnesium sulfate and the solvent removed under reduced pressure. The residue was purified by column chromatography on silica gel, eluting with a mixture of cyclohexane and acetone to afford title compound, 0.1 g.

MS: ESI (+ve) (Method A): 376 (M+H)⁺, Retention time 9.9 min.

¹H NMR (CDCl₃): δ 2.25 (s, 3H), 3.00 (s, 3H), 4.00 (s, 2H), 6.70-6.75 (m, 1H), 6.85-6.90 (m, 1H), 7.00-7.05 (m, 2H), 7.50 (m, 1H), 7.65-7.70 (m, 2H), 7.95 (m, 1H).

### Example 2

### [1-(4-chlorobenzenesulfonyl)-2-methylindolizin-3-yl]acetic acid

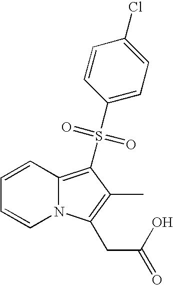

### Preparation 2a

### 2-(4-chlorobenzenesulfonylmethyl)pyridine

A mixture of 4-chlorobenzenesulfonic acid (3.0 g), 2-chloromethylpyridinium chloride (2.5 g), sodium acetate (1.5 g) and n-butanol (10 mL) were heated at 60° C. for 17 hours. The mixture was cooled to room temperature, diluted with saturated aqueous sodium hydrogen carbonate solution and extracted with dichloromethane. The combined extracts were washed with saturated aqueous sodium chloride solution, dried over magnesium sulfate and the solvent removed under reduced pressure. The residue was purified by column chromatography on silica gel, eluting with a mixture of dichloromethane and ethyl acetate to afford title compound as a white solid, 2.7 g.

MS: ESI (+ve) (Method B): 268 (M+H)⁺, Retention time 2.6 min.

### Preparation 2b

### 1-(4-chlorobenzenesulfonyl)-2-methylindolizine

A mixture of 1-bromo-2,2-dimethoxypropane (0.50 mL), butan-2-one (10 mL) and concentrated hydrochloric acid (0.16 mL) was stirred at room temperature for 2 hours. The mixture was treated with potassium carbonate (0.5 g), followed by 2-(4-chlorobenzenesulfonylmethyl)pyridine (0.5 g) and then heated at reflux for 17 hours. The mixture was filtered and the filtrate concentrated under reduced pressure. The residue was triturated with pentane to afford title compound as a brown solid, 0.57 g.

MS: ESI (+ve) (Method B): 306 (M+H)⁺, Retention time 3.6 min.

### Preparation 2c

### [1-(4-chlorobenzenesulfonyl)-2-methylindolizin-3-yl]oxoacetic acid methyl ester

A solution of 1-(4-chlorobenzenesulfonyl)-2-methylindolizine (0.38 g) in tetrahydrofuran at 0° C. was treated with chlorooxoacetic acid methyl ester (0.11 mL) and the resulting solution was stirred at 0° C. for 5 minutes and then at room temperature for 20 hours. The solution was diluted with saturated aqueous sodium hydrogen carbonate solution and extracted with dichloromethane. The combined extracts were washed with saturated aqueous sodium chloride solution, dried over magnesium sulfate and the solvent removed under reduced pressure. The residue was purified by column chromatography on silica gel, eluting with a mixture of dichloromethane and ethyl acetate to afford title compound as a yellow solid, 0.27 g.

MS: ESI (+ve) (Method B): 392 (M+H)⁺, Retention time 3.7 min.

### Preparation 2d

### [1-(4-chlorobenzenesulfonyl)-2-methylindolizin-3-yl]hydroxyacetic acid methyl ester

A mixture of sodium borohydride (0.12 g), methanol (2.0 mL) and water (0.15 mL) at −40° C. was treated with a solution of [1-(4-chlorobenzenesulfonyl)-2-methylindolizin-3-yl]oxoacetic acid methyl ester (0.27 g) in methanol (3.0 mL) and toluene (3.0 mL). The mixture was stirred at −40° C. for 10 minutes and then treated with glacial acetic acid (3.0 mL). The resulting mixture was diluted with saturated aqueous sodium hydrogen carbonate solution and extracted with dichloromethane. The combined extracts were washed with saturated aqueous sodium chloride solution, dried over magnesium sulfate and the solvent removed under reduced pressure. The residue was purified by column chromatography on silica gel, eluting with a mixture of dichloromethane and ethyl acetate to afford title compound, 0.23 g.

MS: ESI (+ve) (Method B): 394 (M+H)⁺, Retention time 3.5 min.

### Preparation 2e

### [1-(4-chlorobenzenesulfonyl)-2-methylindolizin-3-yl]acetic acid methyl ester

A mixture of iodine (0.97 g), triphenylphosphine (0.20 g) and toluene was treated with [1-(4-chlorobenzenesulfonyl)-2-methylindolizin-3-yl]hydroxyacetic acid methyl ester and the resulting mixture was heated at 80° C. for 30 minutes. The mixture was diluted with saturated aqueous sodium chloride solution and extracted with dichloromethane. The combined extracts were washed with saturated aqueous sodium thiosulfate solution, dried over magnesium sulfate and the solvent removed under reduced pressure. The residue was purified by column chromatography on silica gel, eluting with a mixture of dichloromethane and ethyl acetate to afford title compound, 0.13 g.

MS: ESI (+ve) (Method B): 378 (M+H)⁺, Retention time 3.6 min.

### Preparation 2f

### [1-(4-chlorobenzenesulfonyl)-2-methylindolizin-3-yl]acetic acid

A mixture of [1-(4-chlorobenzenesulfonyl)-2-methylindolizin-3-yl]acetic acid methyl ester (0.13 g), tetrahydrofuran (4.0 mL), lithium hydroxide (0.06 g) and water (0.5 mL) was stirred at room temperature for 3 hours. The mixture was acidified by the addition of 0.1 M aqueous hydrochloric acid and extracted with dichloromethane. The combined extracts were dried over magnesium sulfate and the solvent removed under reduced pressure to afford title compound, 0.11 g.

MS: ESI (+ve) (Method A): 364 (M+H)⁺, Retention time 9.9 min.

¹H NMR (DMSO-d6): δ (3H, s), 4.00 (2H, s), 6.95-7.00 (1H, m), 7.25-7.30 (1H, m), 7.60-7.65 (2H, m), 7.85-7.90 (2H, m), 8.10-8.15 (1H, m), 8.25-8.30 (1H, m), 12.65 (br s, 1H).

### Example 3

### (1-benzoyl-2-methylindolizin-3-yl)acetic acid

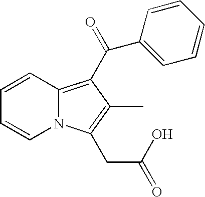

### Preparation 3a

### 1-phenyl-2-pyridin-2-ylethanone

A solution of lithium diisopropylamide (12 mL, 1.8 M in tetrahydrofuran) in tetrahydrofuran (40 mL) at −78° C. was treated dropwise with a solution of 2-methylpyridine (2.0 g) in tetrahydrofuran (5.0 mL). The mixture was stirred at −78° C. for 10 minutes and then a solution of benzonitrile (2.2 mL) in tetrahydrofuran (8.0 mL) was added dropwise. The resulting mixture was stirred at −78° C. for 1 hour and at room temperature for 2 hour and then poured into a saturated aqueous solution of ammonium chloride. The aqueous phase was extracted with diethyl ether and the combined organic phases were extracted with 1.0 M aqueous hydrochloric acid. The aqueous phases were basified with 1.0 M aqueous sodium hydroxide solution and extracted with dichloromethane. The combined organic extracts were washed with saturated aqueous sodium chloride solution, dried over magnesium sulfate and the solvent removed under reduced pressure. The residue was purified by column chromatography on silica gel, eluting with a mixture of dichloromethane and ethyl acetate (1:0 to 5:1 by volume) to afford title compound as a yellow solid, 2.5 g. The NMR showed the product to be a mixture of the keto and enol tautomers.

¹H NMR (CDCl3): δ 4.55 (s), 6.10 (s), 7.00 (dd, J=1.2, 7.4 Hz), 7.10 (dt, J=1.0, 8.1 Hz), 7.20 (dd, J=1.0, 7.5 Hz), 7.35 (d, J=7.9 Hz), 7.40-7.50 (m), 7.55-7.70 (m), 7.85-7.90 (m), 8.10 (m), 8.30 (m), 8.60 (m), 15.50 (s).

### Preparation 3b

### (2-methylindolizin-1-yl)phenylmethanone

A mixture of 1-chloropropan-2-one (2.8 mL), acetone (25 mL), sodium hydrogen carbonate (0.5 g) and 1-phenyl-2-pyridin-2-ylethanone (1.0 g) was heated at reflux for 17 hours. The mixture was filtered and the filtrate concentrated under reduced pressure. The residue was purified by column chromatography on silica gel, eluting with a mixture of dichloromethane and ethyl acetate (1:0 to 5:1 by volume) to afford title compound as a yellow solid, 0.72 g.

¹H NMR (CDCl3): δ 2.2 (s, 3H), 6.60 (dt, J=1.3, 6.8 Hz, 1H), 6.85 (dd, J=1.2, 6.8 Hz, 1H), 7.05 (s, 1H), 7.35-7.45 (m, 4H), 7.60 (m, 2H), 7.85 (dt, J=1.1, 6.8 Hz, 1H).

### Preparation 3c

### (1-benzoyl-2-methylindolizin-3-yl)oxoacetic acid methyl ester

A solution of (2-methylindolizin-1-yl)phenylmethanone (0.29 g) in tetrahydrofuran (4.0 mL) at 0° C. was treated with chlorooxoacetic acid methyl ester (0.15 mL). The resulting mixture was stirred at 0° C. for 5 minutes and then diluted with saturated aqueous sodium hydrogen carbonate solution and extracted with dichloromethane. The combined extracts were washed with saturated aqueous sodium chloride solution, dried over magnesium sulfate and the solvent removed under reduced pressure. The residue was purified by column chromatography on silica gel, eluting with a mixture of dichloromethane and ethyl acetate (1:0 to 4:1 by volume) to afford title compound as a yellow solid, 0.31 g.

¹H NMR (CDCl3): δ 2.30 (s, 3H), 3.95 (s, 3H), 7.10 (dt, J=1.3, 7.0 Hz, 1H), 7.40 (dd, J=1.1, 8.8 Hz, 1H), 7.50 (m, 2H), 7.60 (m, 1H), 7.70 (dt, J=1.1, 8.8 Hz, 1H), 7.75 (m, 2H), 9.95 (dt, J=1.0, 7.0 Hz, 1H).

### Preparation 3d

### (1-benzoyl-2-methylindolizin-3-yl)hydroxyacetic acid methyl ester

A mixture of sodium borohydride (0.090 g), methanol (1.3 mL) and water (0.10 mL) at −40° C. was treated with a solution of (1-benzoyl-2-methylindolizin-3-yl)oxoacetic acid methyl ester (0.17 g) in methanol (2.5 mL) and dichloromethane (0.5 mL). The mixture was stirred at −40° C. for 1 hour and then treated with a mixture of glacial acetic acid (0.2 mL) and water (2.0 mL). The resulting mixture was diluted with saturated aqueous sodium hydrogen carbonate solution and extracted with dichloromethane. The combined extracts were washed with saturated aqueous sodium chloride solution, dried over magnesium sulfate and the solvent removed under reduced pressure. The residue was purified by column chromatography on silica gel, eluting with a mixture of dichloromethane and ethyl acetate (1:0 to 4:1 by volume) to afford title compound, 0.16 g.

MS: ESI (+ve) (Method B): 324 (M+H)⁺, Retention time 2.9 min.

### Preparation 3e

### (1-benzoyl-2-methylindolizin-3-yl)acetic acid methyl ester

A mixture of iodine (0.047 g), triphenylphosphine (0.097 g) and toluene (1.5 mL) was treated with (1-benzoyl-2-methylindolizin-3-yl)hydroxyacetic acid methyl ester and the resulting mixture was heated at 80° C. for 90 minutes. The mixture was diluted with saturated aqueous sodium chloride solution and extracted with dichloromethane. The combined extracts were washed with saturated aqueous sodium thiosulfate solution, dried over magnesium sulfate and the solvent removed under reduced pressure. The residue was purified by column chromatography on silica gel, eluting with a mixture of dichloromethane and ethyl acetate (1:0 to 8.5:1.5 by volume) to afford title compound as a yellow solid, 0.038 g.

MS: ESI (+ve) (Method B): 308 (M+H)⁺, Retention time 3.3 min.

### Preparation 3f

### (1-benzoyl-2-methylindolizin-3-yl)acetic acid

A solution of (1-benzoyl-2-methylindolizin-3-yl)acetic acid methyl ester (0.020 g), methanol (2.0 mL), lithium hydroxide (0.008 g) and water (0.3 mL) was stirred at room temperature for 5 hours. The mixture was acidified by the addition of 0.1 M aqueous hydrochloric acid and extracted with dichloromethane. The combined extracts were dried over magnesium sulfate and the solvent removed under reduced pressure to afford title compound as a yellow/green solid, 0.015 g.

MS: ESI (+ve) (Method A): 294 (M+H)⁺, Retention time 8.8 min.

¹H NMR (DMSO-d6): δ 2.15 (s, 3H), 3.90 (s, 2H), 6.75 (dt, J=1.2, 6.8 Hz, 1H), 6.95 (dd, J=1.0, 9.0 Hz, 1H), 7.30 (dt, J=1.1, 9.0 Hz, 1H), 7.35-7.40 (m, 2H), 7.45-7.50 (m, 3H), 8.10 (dt, J=0.9, 7.0 Hz, 1H).

### Example 4

### [1-(4-fluorobenzenesulfonyl)-2-methylindolizin-3-yl]acetic acid

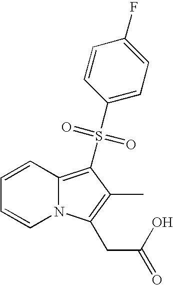

### Preparation 4a

### 2-(4-fluorophenylsulfanylmethyl)pyridine

A mixture of 2-chloromethylpyridine (1.7 g), 4-fluorobenzenethiol (1.1 mL), toluene (20 mL) and 1,8-diazabicyclo[5.4.0]undec-7-ene (3.0 mL) was stirred at room temperature overnight. The mixture was concentrated under reduced pressure and the residue diluted with dichloromethane, washed with saturated aqueous sodium chloride solution and dried over magnesium sulfate. The solvent was removed under reduced pressure and the residue purified by column chromatography on silica gel, eluting with a mixture of cyclohexane and dichloromethane (1:1 to 0:1 by volume) to afford title compound as a pale yellow oil, 1.5 g.

¹H NMR (CDCl₃): δ 4.2 (s, 2H), 6.95 (m, 2H), 7.15 (dd, J=1.0, 7.6 Hz, 1H), 7.25 (d, J=7.8 Hz, 1H), 7.30 (m, 2H), 7.60 (dt, J=1.8, 7.7 Hz, 1H), 8.50 (m, 1H).

### Preparation 4b

### 2-(4-fluorobenzenesulfonylmethyl)pyridine

A mixture of 2-(4-fluorophenylsulfanylmethyl)pyridine (0.93 g), potassium peroxymonosulfate (7.8 g) and dichloromethane (25 mL) was stirred at room temperature for 2 days. The solvent was removed under reduced pressure and the residue purified by column chromatography on silica gel, eluting with a mixture of dichloromethane and ethyl acetate (1:0 to 7:3 by volume) to afford title compound, 0.45 g.

MS: ESI (+ve) (Method B): 252 (M+H)⁺, Retention time 2.7 min.

### Preparation 4c

### 1-(4-fluorobenzenesulfonyl)-2-methylindolizine

A mixture of 1-bromo-2,2-dimethoxypropane (0.19 mL), butan-2-one (8.0 mL) and concentrated hydrochloric acid (0.062 mL) was stirred at room temperature for 1 hour. The resulting solution was treated with potassium carbonate (0.15 g), followed by 2-(4-fluorobenzenesulfonylmethyl)pyridine (0.18 g) and the mixture was heated at 80° C. overnight. The mixture was filtered and the filtrate concentrated under reduced pressure. The residue was purified by column chromatography on silica gel, eluting with a mixture of dichloromethane and ethyl acetate (1:0 to 7:3 by volume) to afford title compound, 0.043 g.

MS: ESI (+ve) (Method B): 290 (M+H)⁺, Retention time 3.6 min.

### Preparation 4d

### [1-(4-fluorobenzenesulfonyl)-2-methylindolizin-3-yl]oxoacetic acid methyl ester

A solution of 1-(4-fluorobenzenesulfonyl)-2-methylindolizine (0.21 g) in tetrahydrofuran (4.0 mL) at room temperature was treated with chlorooxoacetic acid methyl ester (0.10 mL). The resulting mixture was stirred at room temperature overnight and then diluted with saturated aqueous sodium hydrogen carbonate solution and extracted with dichloromethane. The combined extracts were washed with saturated aqueous sodium chloride solution, dried over magnesium sulfate and the solvent removed under reduced pressure. The residue was purified by column chromatography on silica gel, eluting with a mixture of dichloromethane and ethyl acetate (1:0 to 9:1 by volume) to afford title compound, 0.030 g.

MS: ESI (+ve) (Method B): 376 (M+H)⁺, Retention time 3.5 min.

### Preparation 4e

### [1-(4-fluorobenzenesulfonyl)-2-methylindolizin-3-yl]hydroxyacetic acid methyl ester

A mixture of sodium borohydride (0.042 g), methanol (2.0 mL) and water (0.10 mL) at −40° C. was treated with a solution of [1-(4-fluorobenzenesulfonyl)-2-methylindolizin-3-yl]oxoacetic acid methyl ester (0.092 g) in methanol (2.0 mL) and dichloromethane (0.5 mL). The mixture was stirred at −40° C. for 10 minutes and then treated with a mixture of glacial acetic acid (0.3 mL) and water (2.7 mL). The resulting mixture was diluted with saturated aqueous sodium hydrogen carbonate solution and extracted with dichloromethane. The combined extracts were washed with saturated aqueous sodium chloride solution, dried over magnesium sulfate and the solvent removed under reduced pressure. The residue was purified by column chromatography on silica gel, eluting with a mixture of dichloromethane and ethyl acetate (1:0 to 9:1 by volume) to afford title compound, 0.080 g.

MS: ESI (+ve) (Method B): 378 (M+H)⁺, Retention time 3.0 min.

### Preparation 4f

### [1-(4-fluorobenzenesulfonyl)-2-methylindolizin-3-yl]acetic acid methyl ester

A mixture of iodine (0.054 g), triphenylphosphine (0.11 g) and toluene (2.0 mL) was treated with a solution of [1-(4-fluorobenzenesulfonyl)-2-methylindolizin-3-yl]hydroxyacetic acid methyl ester (0.080 g) in toluene (2.0 mL) and the resulting mixture was heated at 80° C. for 90 minutes. The mixture was diluted with saturated aqueous sodium chloride solution and extracted with dichloromethane. The combined extracts were washed with saturated aqueous sodium thiosulfate solution, dried over magnesium sulfate and the solvent removed under reduced pressure. The residue was purified by column chromatography on silica gel, eluting with a mixture of dichloromethane and ethyl acetate (1:0 to 9:1 by volume) to afford title compound, 0.061 g.

MS: ESI (+ve) (Method B): 362 (M+H)⁺, Retention time 3.5 min.

### Preparation 4 g

### [1-(4-fluorobenzenesulfonyl)-2-methylindolizin-3-yl]acetic acid

A mixture of [1-(4-fluorobenzenesulfonyl)-2-methylindolizin-3-yl]acetic acid methyl ester (0.064 g), methanol (5.0 mL), water (1.0 mL) and 5.0 M aqueous sodium hydroxide solution (0.36 mL) was stirred at room temperature for 5 hours. The pH of the mixture was adjusted to 5 by the addition of glacial acetic acid and then concentrated under reduced pressure. The residue was purified by preparative reverse-phase HPLC using a gradient over 30 minutes of acetonitrile in water (30% to 90% of organic modifier) to afford title compound as an off-white solid.

MS: ESI (+ve) (Method A): 348 (M+H)⁺, Retention time 9.1 min.

¹H NMR (DMSO-d6): δ 2.25 (s, 3H), 3.90 (s, 2H), 6.90 (dt, J=1.1, 6.9 Hz, 1H), 7.20 (dd, J=0.8, 9.1 Hz, 1H), 7.30 (m, 2H), 7.90 (m, 2H), 8.05 (dt, J=1.1, 9.1 Hz, 1H), 8.20 (dt, J=0.8, 6.9 Hz, 1H), 12.55 (br s, 1H).

### Example 5

### [1-(benzothiazol-2-ylsulfanyl)-2-methylindolizin-3-yl]acetic acid

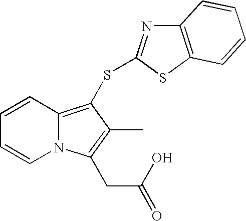

### Preparation 5a

### [1-(benzothiazol-2-ylsulfanyl)-2-methylindolizin-3-yl]acetic acid ethyl ester

A mixture of (2-methylindolizin-3-yl)acetic acid ethyl ester (0.52 g), 2,2′-dithiobis(benzothiazole) (0.79 g) and ethanol (25 mL) was heated a reflux for 2.5 hours. The solvent was removed under reduced pressure and the residue dissolved in ethyl acetate, washed with saturated aqueous sodium carbonate solution and dried over magnesium sulfate. The solvent was removed under reduced pressure and the residue purified by column chromatography on silica gel, eluting with dichloromethane to afford title compound, 0.48 g.

MS: ESI (+ve) (Method B): 383 (M+H)⁺, Retention time 4.4 min.

### Preparation 5b

### [1-(benzothiazol-2-ylsulfanyl)-2-methylindolizin-3-yl]acetic acid

A mixture of [1-(benzothiazol-2-ylsulfanyl)-2-methylindolizin-3-yl]acetic acid ethyl ester (0.390 g), methanol (10 mL), water (2.0 mL) and 5.0 M aqueous sodium hydroxide solution (5.3 mL) was stirred at room temperature for 4 hours. The pH of the mixture was adjusted to 1 by the addition of 1.0 M aqueous hydrochloric acid and then concentrated under reduced pressure. The residue was purified by preparative reverse-phase HPLC using a gradient over 30 minutes of acetonitrile in water (30% to 90% of organic modifier) to afford title compound as a pale green solid, 0.004 g.

MS: ESI (+ve) (Method A): 355 (M+H)⁺, Retention time 10.9 min.

¹H NMR (DMSO-d6): δ 2.25 (s, 3H), 4.05 (s, 2H), 6.80 (dt, J=1.3, 7.0 Hz, 1H), 7.00 (dd, J=0.8, 8.8 Hz, 1H), 7.20 (m, 1H), 7.35 (m, 1H), 7.55 (m, 1H), 7.75 (m, 2H), 8.20 (dt, J=0.8, 7.0 Hz, 1H), 12.55 (br s, 1H).

### Example 6

### [6-cyano-1-(4-methanesulfonylphenylsulfanyl)-2-methylindolizin-3-yl]acetic acid

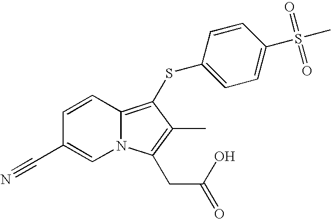

### Preparation 6a

### 2-methylindolizine-6-carbonitrile

A mixture of 1-bromopropan-2-one (1.5 g), acetonitrile (5.0 mL), sodium hydrogen carbonate (1.1 g) and nicotinonitrile (0.5 g) was heated at reflux for 24 hours. The mixture was diluted with water, extracted with ethyl acetate and the combined extracts dried over magnesium sulfate. The solvent was removed under reduced pressure and the residue purified by column chromatography on silica gel, eluting with a mixture of cyclohexane and ethyl acetate (7:3 by volume) to afford title compound, 0.33 g.

¹H NMR (CDCl₃): δ 2.30 (s, 3H), 6.40 (br s, 1H), 6.65 (dd, J=1.4, 9.2 Hz, 1H), 7.20 (br s, 1H), 7.30 (d, J=9.2 Hz, 1H), 8.25 (m, 1H).

### Preparation 6b

### (6-cyano-2-methylindolizin-3-yl)acetic acid ethyl ester

To a solution of 2-methylindolizine-6-carbonitrile (0.17 g) in toluene (20 mL) at reflux was added ethyl diazoacetate (0.12 mL) portionwise, each addition was followed by a small portion of copper powder (total amount 0.060 g). The resulting mixture was heated at reflux for 4 hours and then the solvent removed under reduced pressure. The residue was purified by column chromatography on silica gel, eluting with a mixture of cyclohexane and dichloromethane (4:6 by volume) to afford title compound, 0.095 g.

MS: ESI (+ve) (Method B): 243 (M+H)⁺, Retention time 3.5 min.

### Preparation 6c

### bis[4-(methylsulfonyl)phenyl]disulfide

A mixture of 1-fluoro-4-methanesulfonylbenzene (32 g), sodium hydrogensulfide (64 g) and 1-methylpyrrolidin-2-one (100 mL) was stirred at 80° C. for 60 minutes and then at room temperature for 60 minutes. The mixture was diluted with water, filtered and the filtrate acidified by the addition of concentrated hydrochloric acid. The resulting precipitate was collected by filtration, washed with water and dried to afford title compound as a white solid, 15 g.

MS: ESI (+ve) (Method B): 375 (M+H)⁺, Retention time 3.3 min.

### Preparation 6d

### [6-cyano-1-(4-methanesulfonylphenylsulfanyl)-2-methylindolizin-3-yl]acetic acid ethyl ester

Sulfuryl chloride (0.017 mL) was added to a mixture of bis[4-(methylsulfonyl)phenyl]disulfide (0.090 g) in 1,2-dichloroethane (2.5 mL) at 0° C. and the resulting mixture was stirred at room temperature for 1 hour. A solution of (6-cyano-2-methylindolizin-3-yl)acetic acid ethyl ester (0.039 g) in 1,2-dichloroethane (2.5 mL) was added and the resulting mixture was stirred at room temperature overnight. The solvent was removed under reduced pressure and the residue purified by column chromatography on silica gel, eluting with a mixture of dichloromethane and ethyl acetate (19:1 by volume) to afford title compound.

MS: ESI (+ve) (Method B): 429 (M+H)⁺, Retention time 3.8 min.

### Preparation 6e

### [6-cyano-1-(4-methanesulfonylphenylsulfanyl)-2-methylindolizin-3-yl]acetic acid

A mixture of [6-cyano-1-(4-methanesulfonylphenylsulfanyl)-2-methylindolizin-3-yl]acetic acid ethyl ester (0.12 g), tetrahydrofuran (5.0 mL) and 1.0 M aqueous lithium hydroxide solution (2.0 mL) was stirred at room temperature for 2 hours. The mixture was concentrated under reduced pressure, acidified by the addition sodium dihydrogenphosphate monohydrate and then extracted with ethyl acetate. The combined extracts were dried over magnesium sulfate and the solvent removed under reduced pressure. The residue was triturated with a mixture of ethyl acetate and diethyl ether to afford title compound as a light brown solid, 0.084 g.

MS: ESI (+ve) (Method A): 401 (M+H)⁺, Retention time 9.2 min.

¹H NMR (DMSO-d6): δ 2.15 (s, 3H), 3.15 (s, 3H), 4.15 (s, 2H), 7.10 (m, 3H), 7.55 (dd, J=1.0, 9.2 Hz, 1H), 7.75 (d, J=8.7 Hz, 2H), 9.15 (t, J=1.0 Hz, 1H).

### Example 7

### [7-cyano-1-(4-methanesulfonylphenylsulfanyl)-2-methylindolizin-3-yl]acetic acid

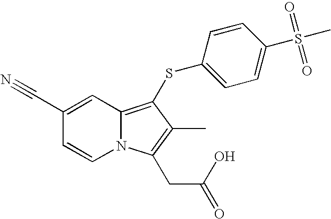

### Preparation 7a

### 2-methylisonicotinonitrile

A mixture of 2-chloroisonicotinonitrile (28 g), tetrakis(triphenylphosphine)palladium(0) (5.0 g), trimethylaluminum (2.0 M in hexanes, 110 mL) and 1,4-dioxane (400 mL) was heated at reflux for 2 hours. The mixture was cooled to room temperature, diluted with 1.0 M aqueous hydrochloric acid and the organic phase extracted with 1.0 M aqueous hydrochloric acid. The combined aqueous phases were washed with diethyl ether, basified by the addition of concentrated aqueous sodium hydroxide solution and then extracted with diethyl ether. The combined extracts were dried over magnesium sulfate and the solvent removed under reduced pressure to afford title compound, 24 g.

¹H NMR (CDCl₃): δ 2.65 (s, 3H), 7.35 (m, 1H), 7.40 (br s, 1H), 8.70 (d, J=5.0 Hz, 1H).

### Preparation 7b

### 2-methylindolizine-7-carbonitrile

A mixture of 2-methylisonicotinonitrile (4.0 g), 1-bromopropan-2-one (9.3 g), sodium hydrogen carbonate (6.8 g) and acetonitrile (40 mL) was heated at reflux for 14 hours. The mixture was cooled to room temperature, diluted with water and extracted with ethyl acetate. The combined extracts were washed with saturated aqueous sodium chloride solution, dried over magnesium sulfate and the solvent removed under reduced pressure. Purification of the residue by column chromatography on silica gel, eluting with a mixture of cyclohexane and ethyl acetate gave title compound as a yellow solid, 1.5 g.

¹H NMR (CDCl₃): δ 2.35 (s, 3H), 6.50 (dd, J=1.6, 7.2 Hz, 1H), 6.55 (br s, 1H), 7.25 (br s, 1H), 7.70 (br s, 1H), 7.80 (m, 1H).

### Preparation 7c

### (7-cyano-2-methylindolizin-3-yl)acetic acid ethyl ester

The title compound was prepared by the method of Preparation 6b using 2-methylindolizine-7-carbonitrile.

¹H NMR (CDCl₃): δ 1.25 (t, J=7.1 Hz, 3H), 2.35 (s, 3H), 3.90 (s, 2H), 4.15 (q, J=7.1 Hz, 2H), 6.55 (m, 1H), 6.60 (d, J=1.7 Hz, 1H), 7.70 (br s, 1H), 7.85 (d, J=7.1 Hz, 1H).

### Preparation 7d

### [7-cyano-1-(4-methanesulfonylphenylsulfanyl)-2-methylindolizin-3-yl]acetic acid ethyl ester

The title compound was prepared by the method of Preparation 6d using (7-cyano-2-methylindolizin-3-yl)acetic acid ethyl ester and bis[4-(methylsulfonyl)phenyl]disulfide.

MS: ESI (−ve) (Method B): 428 (M−H)⁻, Retention time 3.7 min.

### Preparation 7e

### [7-cyano-1-(4-methanesulfonylphenylsulfanyl)-2-methylindolizin-3-yl]acetic acid

A mixture of [7-cyano-1-(4-methanesulfonylphenylsulfanyl)-2-methylindolizin-3-yl]acetic acid ethyl ester (0.070 g), tetrahydrofuran (4.0 mL), water (4.0 mL) and lithium hydroxide (0.021 g) was stirred at room temperature for 1 hour. The mixture was acidified by the addition of 1.0 M aqueous hydrochloric acid and extracted with ethyl acetate. The combined extracts were dried over magnesium sulfate and the solvent removed under reduced pressure. The residue was purified by preparative reverse-phase HPLC using a gradient of acetonitrile in water to afford title compound as an orange solid, 0.005 g.

MS: ESI (+ve) (Method A): 401 (M+H)⁺, Retention time 9.2 min.

¹H NMR (DMSO-d6): δ 2.25 (s, 3H), 3.05 (s, 3H), 4.10 (s, 2H), 6.85 (dd, J=1.8, 7.3 Hz, 1H), 7.15 (d, J=8.7 Hz, 2H), 7.75 (d, J=8.7 Hz, 2H), 7.95 (dd, J=0.9, 1.8 Hz, 1H), 8.25 (dd, J=0.9, 7.3 Hz, 1H).

### Example 8

### {7-cyano-2-methyl-1-[4-(morpholine-4-sulfonyl)phenylsulfanyl]indolizine-3-yl}acetic acid

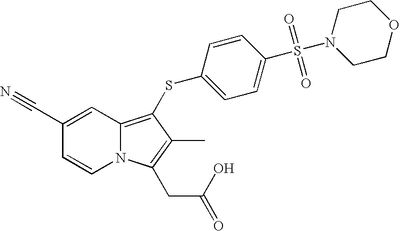

### Preparation 8a

### 4-(4-fluorobenzenesulfonyl)morpholine

A solution of 4-fluorobenzenesulfonyl chloride (6.0 g) in dichloromethane (40 mL) was added dropwise over a period of 10 minutes to a solution of morpholine (8.0 mL) in dichloromethane (40 mL) at 0° C. and the resulting mixture was stirred at 0° C. for 10 minutes and then at room temperature for 10 minutes. The mixture was washed with water, dried over magnesium sulfate and solvent removed under reduced pressure to afford title compound as a white solid, 7.5 g.

¹H NMR (CDCl₃): δ 3.00 (t, J=4.9 Hz, 4H), 3.75 (t, J=4.9 Hz, 4H), 7.25 (t, J=8.4 Hz, 2H), 7.80 (m, 2H).

### Preparation 8b

### bis[4-(morpholine-4-sulfonyl)benzene]disulfide

A mixture of 4-(4-fluorobenzenesulfonyl)morpholine (0.5 g), sodium hydrogensulfide (1.5 g) and 1-methylpyrrolidin-2-one (2.0 mL) was stirred at 80° C. for 90 minutes and then at room temperature for 5 hours. The mixture was filtered and the filtrated extracted with ethyl acetate. The aqueous phase was acidified by the addition of concentrated hydrochloric acid, extracted with ethyl acetate and the combined extracts dried over magnesium sulphate. The solvent was removed under reduced pressure and the residue triturated with water to afford title compound as an off-white solid, 0.56 g.

¹H NMR (CDCl₃): δ 3.00 (t, J=4.7 Hz, 8H), 3.75 (t, J=4.7 Hz, 8H), 7.65 (d, J=8.6 Hz, 4H), 7.70 (d, J=8.6 Hz, 4H).

MS: ESI (+ve) (Method B): 517 (M+H)⁺, Retention time 3.6 min.

### Preparation 8c

### {7-cyano-2-methyl-1-[4-(morpholine-4-sulfonyl)phenylsulfanyl]indolizin-3-yl}acetic acid ethyl ester

The title compound was prepared by the method of Preparation 6d using (7-cyano-2-methylindolizin-3-yl)acetic acid ethyl ester and bis[4-(morpholine-4-sulfonyl)benzene]disulfide.

MS: ESI (+ve) (Method B): 500 (M+H)⁺, Retention time 4.0 min.

### Preparation 8d

### {7-cyano-2-methyl-1-[4-(morpholine-4-sulfonyl)phenylsulfanyl]indolizin-3-yl}acetic acid

A mixture of {7-cyano-2-methyl-1-[4-(morpholine-4-sulfonyl)phenylsulfanyl]indolizin-3-yl}acetic acid ethyl ester (0.16 g), methanol (10 mL), water (2.0 mL) and 5.0 M aqueous sodium hydroxide solution (0.33 mL) was stirred at room temperature for 30 minutes. The mixture was acidified by the addition of glacial acetic acid and then concentrated under reduced pressure. The residue was purified by preparative reverse-phase HPLC using a gradient over 30 minutes of acetonitrile in water (50% to 95% of organic modifier) to afford title compound as a yellow solid, 0.035 g.

MS: ESI (+ve) (Method A): 472 (M+H)⁺, Retention time 12.5 min.

¹H NMR (DMSO-d6): δ 2.20 (s, 3H), 2.80 (m, 4H), 3.60 (m, 4H), 4.15 (s, 2H), 7.00 (dd, J=1.8, 7.3 Hz, 1H), 7.10 (d, J=8.7 Hz, 2H), 7.55 (d, J=8.7 Hz, 2H), 8.10 (dd, J=0.9, 1.8 Hz, 1H), 8.40 (dd, J=0.9, 7.3 Hz, 1H), 12.7 (br s, 1H).

### Example 9

### [7-cyano-2-methyl-1-(4-methylsulfamoylphenylsulfanyl)indolizin-3-yl]acetic acid

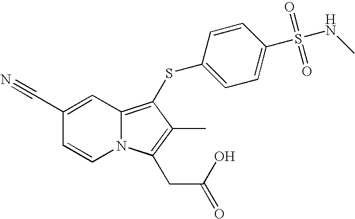

### Preparation 9a

### 4-fluoro-N-methylbenzenesulfonamide

A solution of 4-fluorobenzenesulfonyl chloride (9.0 g) in dichloromethane (50 mL) was added dropwise over a period of 10 minutes to a mixture methylamine (10 mL, 40% wt. in water) in dichloromethane (50 mL) at 0° C. and the resulting mixture was stirred at 0° C. for 10 minutes and then at room temperature for 10 minutes. The mixture was washed with water, dried over magnesium sulfate and solvent removed under reduced pressure to afford title compound as a white solid, 8.2 g.

¹H NMR (CDCl₃): δ 2.65 (d, J=5.2 Hz, 3H), 4.65 (m, 1H), 7.20 (m, 2H), 7.90 (m, 2H).

### Preparation 9b

### 4,4′-dithiobis(N-methylbenzenesulfonamide)

A mixture of 4-fluoro-N-methylbenzenesulfonamide (0.5 g), sodium hydrogensulfide (2.0 g) and 1-methylpyrrolidin-2-one (2.5 mL) was stirred at 80° C. for 90 minutes and then at room temperature for 4 hours. The mixture was diluted with water, acidified by the addition of concentrated hydrochloric acid and extracted with dichloromethane. The combined extracts were dried over magnesium sulfate and the solvent removed under reduced pressure. The residue was triturated with water to afford title compound as a white solid, 0.20 g.

MS: ESI (−ve) (Method B): 403 (M−H)⁻, Retention time 3.2 min.

### Preparation 9c

### [7-cyano-2-methyl-1-(4-methylsulfamoylphenylsulfanyl)indolizin-3-yl]acetic acid ethyl ester

The title compound was prepared by the method of Preparation 6d using (7-cyano-2-methylindolizin-3-yl)acetic acid ethyl ester and 4,4′-dithiobis(N-methylbenzenesulfonamide).

MS: ESI (+ve) (Method B): 444 (M+H)⁺, Retention time 3.8 min.

### Preparation 9d

### [7-cyano-2-methyl-1-(4-methylsulfamoylphenylsulfanyl)indolizin-3-yl]acetic acid

A mixture of [7-cyano-2-methyl-1-(4-methylsulfamoylphenylsulfanyl)indolizin-3-yl]acetic acid ethyl ester (0.11 g), methanol (10 mL), water (2.0 mL) and 5.0 M aqueous sodium hydroxide solution (0.33 mL) was stirred at room temperature for 30 minutes. The mixture was acidified by the addition of glacial acetic acid and then concentrated under reduced pressure. The residue was purified by preparative reverse-phase HPLC using a gradient over 30 minutes of acetonitrile in water (50% to 90% of organic modifier) to afford title compound as a yellow solid, 0.030 g.

MS: ESI (+ve) (Method A): 416 (M+H)⁺, Retention time 9.7 min.

¹H NMR (DMSO-d6): δ 2.20 (s, 3H), 2.35 (d, J=5.0 Hz, 3H), 4.15 (s, 2H), 7.00 (dd, J=1.8, 7.3 Hz, 1H), 7.10 (d, J=8.7 Hz, 2H), 7.35 (q, J=5.0 Hz, 1H), 7.60 (d, J=8.7 Hz, 2H), 8.10 (dd, J=0.9, 1.8 Hz, 1H), 8.40 (dd, J=0.9, 7.3 Hz, 1H), 12.6 (br s, 1H).

### Example 10

### [7-cyano-1-(4-ethanesulfonylaminophenylsulfanyl)-2-methylindolizin-3-yl]acetic acid

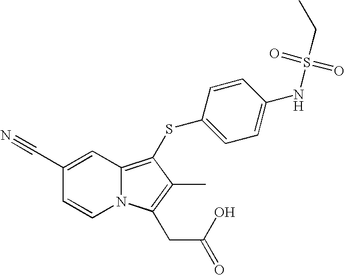

### Preparation 10a

### ethanesulfonic acid [4-(4-ethanesulfonylaminophenyl-disulfanyl)phenyl]amide

A solution of ethanesulfonyl chloride (1.9 mL) in dichloromethane (5.0 mL) was added dropwise to a solution of 4-aminophenyl disulfide (2.0 g) and triethylamine (4.5 mL) in dichloromethane (20 mL) at −40° C. and the resulting mixture was warmed to room temperature over a period of 4 hours. The mixture was washed with 1.0 M aqueous sodium hydroxide solution and the aqueous phase acidified by the addition of 1.0 M aqueous hydrochloric acid and then extracted with ethyl acetate. The combined extracts were dried over magnesium sulfate and solvent removed under reduced pressure to afford title compound as a white solid, 2.0 g.

¹H NMR (DMSO-d6): δ 1.15 (t, J=7.4 Hz, 6H), 3.10 (q, J=7.4 Hz, 4H), 7.20 (m, 4H), 7.45 (m, 4H), 9.85 (br s, 2H).

### Preparation 10b

### [7-cyano-1-(4-ethanesulfonylaminophenylsulfanyl)-2-methylindolizin-3-yl]acetic acid ethyl ester

The title compound was prepared by the method of Preparation 6d using (7-cyano-2-methylindolizin-3-yl)acetic acid ethyl ester and ethanesulfonic acid [4-(4-ethanesulfonylaminophenyldisulfanyl)phenyl]amide.

MS: ESI (+ve) (Method B): 458 (M+H)⁺, Retention time 3.8 min.

### Preparation 10c

### [7-cyano-1-(4-ethanesulfonylaminophenylsulfanyl)-2-methylindolizin-3-yl]acetic acid

A mixture of [7-cyano-1-(4-ethanesulfonylaminophenylsulfanyl)-2-methylindolizin-3-yl]acetic acid ethyl ester (0.23 g), methanol (8.0 mL) and 1.0 M aqueous sodium hydroxide solution (3.5 mL) was stirred at room temperature for 2 hours. The mixture was concentrated under reduced pressure, diluted with water and washed with dichloromethane. The aqueous phase was acidified by the addition of glacial acetic acid and extracted with dichloromethane. The combined extracts were washed with saturated aqueous sodium chloride solution, dried over magnesium sulfate and the solvent removed under reduced pressure. The residue was purified by preparative reverse-phase HPLC using a gradient over 35 minutes of acetonitrile in water (40% to 75% of organic modifier) to afford title compound as a yellow solid, 0.042 g.

MS: ESI (+ve) (Method A): 430 (M+H)⁺, Retention time 9.6 min.

¹H NMR (DMSO-d6): δ 1.30 (t, J=7.4 Hz, 3H), 2.30 (s, 3H), 3.05 (q, J=7.4 Hz, 2H), 3.95 (s, 2H), 6.25 (br s, 1H), 6.70 (dd, J=1.7, 7.2 Hz, 1H), 6.90 (d, J=8.7 Hz, 2H), 7.00 (d, J=8.7 Hz, 2H), 7.90 (d, J=7.4 Hz, 1H), 7.95 (s, 1H).

### Example 11

### {7-cyano-1-[2-fluoro-4-(morpholine-4-sulfonyl)phenylsulfanyl]-2-methyl-1-indolizin-3-yl}acetic acid

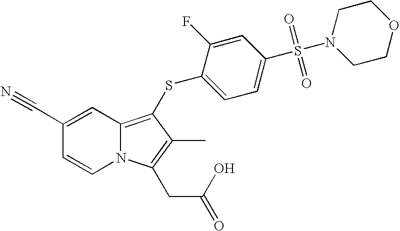

### Preparation 11a

### 4-(3,4-difluorobenzenesulfonyl)morpholine

A solution of 3,4-difluorobenzenesulfonyl chloride (5.0 g) in dichloromethane (20 mL) was added dropwise to a solution of morpholine (6.1 mL) in dichloromethane (30 mL) at 0° C. and the resulting mixture was stirred at 0° C. for 15 minutes and then at room temperature for 20 minutes. The mixture was washed with water, dried over magnesium sulfate and solvent removed under reduced pressure to afford title compound as a white solid, 6.4 g.

¹H NMR (CDCl₃): δ 3.05 (m, 4H), 3.75 (m, 4H), 7.35-7.40 (m, 1H), 7.55 (m, 1H), 7.60 (m, 1H).

### Preparation 11b

### bis[2-fluoro-4-(morpholine-4-sulfonyl)benzene]disulfide

A mixture of 4-(3,4-difluorobenzenesulfonyl)morpholine (1.0 g), sodium hydrogensulfide (2.9 g) and 1-methylpyrrolidin-2-one (4.0 mL) was stirred at 80° C. for 90 minutes and then at room temperature for 5 hours. The mixture was diluted with water, washed with ethyl acetate and the aqueous phase acidified by the addition of concentrated hydrochloric acid. The mixture was extracted with ethyl acetate and the combined extracts dried over magnesium sulfate and the solvent removed under reduced pressure. The residue was triturated with water to afford title compound as a white solid, 0.94 g.

MS: ESI (+ve) (Method B): 553 (M+H)⁺, Retention time 3.1 min.

### Preparation 11c

### {7-cyano-1-[2-fluoro-4-(morpholine-4-sulfonyl)phenylsulfanyl]-2-methyl-1-indolizin-3-yl}acetic acid ethyl ester

The title compound was prepared by the method of Preparation 6d using (7-cyano-2-methylindolizin-3-yl)acetic acid ethyl ester and bis[2-fluoro-4-(morpholine-4-sulfonyl)benzene]disulfide.

MS: ESI (+ve) (Method B): 518 (M+H)⁺, Retention time 4.0 min.

### Preparation 11d

### {7-cyano-1-[2-fluoro-4-(morpholine-4-sulfonyl)phenylsulfanyl]-2-methyl-1-indolizin-3-yl}acetic acid

A mixture of {7-cyano-1-[2-fluoro-4-(morpholine-4-sulfonyl)phenylsulfanyl]-2-methyl-1-indolizin-3-yl}acetic acid ethyl ester (0.28 g), methanol (8.0 mL) and 1.0 M aqueous sodium hydroxide solution (3.5 mL) was stirred at room temperature for 2 hours. The mixture was concentrated under reduced pressure, diluted with water and washed with dichloromethane. The aqueous phase was acidified by the addition of glacial acetic acid and extracted with dichloromethane. The combined extracts were washed with saturated aqueous sodium chloride solution, dried over magnesium sulfate and the solvent removed under reduced pressure. The residue was purified by preparative reverse-phase HPLC using a gradient over 35 minutes of acetonitrile in water (40% to 75% of organic modifier) to afford title compound as a yellow solid, 0.071 g.

MS: ESI (+ve) (Method A): 490 (M+H)⁺, Retention time 10.8 min.

¹H NMR (CDCl₃): δ 2.25 (s, 3H), 2.95 (m, 4H), 3.70 (m, 4H), 4.00 (s, 2H), 6.50 (dd, J=7.5, 8.0 Hz, 1H), 6.80 (dd, J=1.7, 7.2 Hz, 1H), 7.20 (dd, J=1.7, 8.3 Hz, 1H), 7.40 (dd, J=1.7, 9.1 Hz, 1H), 7.95 (m 1H), 8.00 (d, J=7.2 Hz, 1H).

### Example 12

### [7-cyano-1-(4-cyclopropylsulfamoylphenylsulfanyl)-2-methylindolizin-3-yl]acetic acid

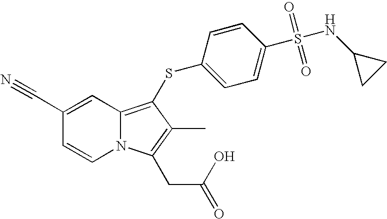

### Preparation 12a

### 4,4′-dithiobisbenzenesulfonic acid, disodium salt

Prepared by the method of Smith et al. (J. Org. Chem., 1964, 29, 1484-1488).

### Preparation 12b

### 4,4′-dithiobisbenzenesulfonyl chloride

A mixture of 4,4′-dithiobisbenzenesulfonic acid, disodium salt (6.2 g), phosphorus oxychloride (6.2 mL) and phosphorus pentachloride (3.1 g) was heated at reflux for 2 hours. The mixture was cooled to room temperature, diluted with dichloromethane and poured into ice. The organic phase was washed with saturated aqueous sodium hydrogen carbonate solution, dried over magnesium sulfate and the solvent removed under reduced pressure. The residue was triturated with cyclohexane to afford title compound as a light brown solid, 3.5 g.

¹H NMR (CDCl₃): δ 7.70 (m, 4H), 8.00 (m, 4H).

### Preparation 12c

### bis[(cyclopropyl-4-sulfonyl)benzene]disulfide

A solution of 4,4′-dithiobisbenzenesulfonyl chloride (1.0 g) in dichloromethane (20 mL) was added dropwise to a mixture of cyclopropylamine (0.4 mL), triethylamine (1.6 mL) and dichloromethane (15 mL) at 0° C. and the resulting mixture was stirred at room temperature for 2 days. The mixture was concentrated under reduced pressure and the residue was purified by column chromatography on silica gel, eluting with a mixture of dichloromethane and methanol (1:0 to 99:1 by volume) to afford title compound as a pale yellow solid, 1.1 g.

MS: ESI (+ve) (Method B): 457 (M+H)⁺, Retention time 3.7 min.

### Preparation 12d

### [7-cyano-1-(4-cyclopropylsulfamoylphenylsulfanyl)-2-methylindolizin-3-yl]acetic acid ethyl ester

The title compound was prepared by the method of Preparation 6d using (7-cyano-2-methylindolizin-3-yl)acetic acid ethyl ester and bis[(cyclopropyl-4-sulfonyl)benzene]disulfide.

MS: ESI (+ve) (Method B): 470 (M+H)⁺, Retention time 4.0 min.

### Preparation 12e

### [7-cyano-1-(4-cyclopropylsulfamoylphenylsulfanyl)-2-methylindolizin-3-yl]acetic acid

A mixture of [7-cyano-1-(4-cyclopropylsulfamoylphenylsulfanyl)-2-methylindolizin-3-yl]acetic acid ethyl ester (0.19 g), methanol (8.0 mL) and 1.0 M aqueous sodium hydroxide solution (3.5 mL) was stirred at room temperature for 2 hours. The mixture was concentrated under reduced pressure, diluted with water and washed with dichloromethane. The aqueous phase was acidified by the addition of glacial acetic acid and extracted with dichloromethane. The combined extracts were washed with saturated aqueous sodium chloride solution, dried over magnesium sulfate and the solvent removed under reduced pressure. The residue was purified by preparative reverse-phase HPLC using a gradient over 35 minutes of acetonitrile in water (40% to 75% of organic modifier) to afford title compound as a yellow solid, 0.036 g.

MS: ESI (+ve) (Method A): 442 (M+H)⁺, Retention time 10.4 min.

¹H NMR (CDCl₃): δ 0.55 (m, 4H), 2.20 (m, 1H), 2.25 (s, 3H), 4.00 (s, 2H), 4.95 (s, 1H), 6.75 (dd, J=1.7, 7.2 Hz, 1H), 7.00 (d, J=8.6 Hz, 2H), 7.65 (d, J=8.6 Hz, 2H), 7.90 (dd, J=0.8, 1.7 Hz, 1H), 7.95 (dd, J=0.8, 7.2 Hz, 1H).

### Example 13

### [1-(3-chloro-4-methanesulfonylphenylsulfanyl)-7-cyano-2-methylindolizin-3-yl]acetic acid

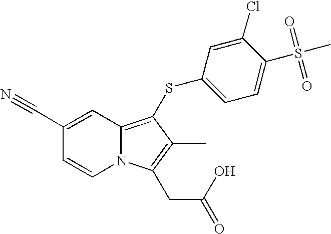

### Preparation 13a

### 2-chloro-4-fluoro-1-methanesulfonylbenzene

3-Chloroperoxybenzoic acid (32 g) was added portionwise to a solution of 2-chloro-4-fluoro-1-methylsulfanylbenzene (11.5 g) in dichloromethane (300 mL) at room temperature and the resulting mixture was stirred at room temperature for 2 hours. The mixture was filtered and the filtrated washed with saturated aqueous sodium thiosulfate solution and saturated aqueous sodium hydrogen carbonate solution and then dried over magnesium sulfate. The solvent was removed under reduced pressure and the residue triturated with diethyl ether to afford title compound as a sandy solid, 8.4 g.

¹H NMR (CDCl₃): δ 3.25 (s, 3H), 7.20 (m, 1H), 7.30 (dd, J=2.4, 8.2, 1H), 8.20 (dd, J=5.9, 8.9 Hz, 1H).

### Preparation 13b

### bis(3-chloro-4-methanesulfonylbenzene)disulfide

A mixture of 2-chloro-4-fluoro-1-methanesulfonylbenzene (1.0 g), sodium hydrogensulfide (3.6 g) and 1-methylpyrrolidin-2-one (5.0 mL) was stirred at 80° C. for 90 minutes and then at room temperature for 4 hours. The mixture was diluted with water (40 mL), acidified by the addition of concentrated hydrochloric acid and extracted with dichloromethane. The combined extracts were dried over magnesium sulfate and the solvent removed under reduced pressure. The residue was triturated with water and the resulting solid crystallised from toluene to afford title compound as a white crystalline solid, 0.69 g.

¹H NMR (DMSO-d6): δ 3.35 (s, 6H), 7.80 (dd, J=2.0, 8.4 Hz, 2H), 7.95 (d, J=2.0 Hz, 2H), 8.05 (d, J=8.4 Hz, 2H).

MS: ESI (+ve) (Method B): 443 (M+H)⁺, Retention time 3.6 min.

### Preparation 13c

### [1-(3-chloro-4-methanesulfonylphenylsulfanyl)-7-cyano-2-methylindolizin-3-yl]acetic acid ethyl ester

The title compound was prepared by the method of Preparation 6d using (7-cyano-2-methylindolizin-3-yl)acetic acid ethyl ester and bis(3-chloro-4-methanesulfonylbenzene)disulfide.

MS: ESI (+ve) (Method B): 463 (M+H)⁺, Retention time 4.0 min.

### Preparation 13d

### [1-(3-chloro-4-methanesulfonylphenylsulfanyl)-7-cyano-2-methylindolizin-3-yl]acetic acid

A mixture of [1-(3-chloro-4-methanesulfonylphenylsulfanyl)-7-cyano-2-methylindolizin-3-yl]acetic acid ethyl ester (0.30 g), tetrahydrofuran (20 mL), water (20 mL) and lithium hydroxide (0.080 g) was stirred at room temperature for 2 hours. The mixture was acidified by the addition of 1.0 M aqueous hydrochloric acid and extracted with ethyl acetate. The combined extracts were dried over magnesium sulfate and the solvent removed under reduced pressure. The residue was purified by preparative reverse-phase HPLC using a gradient of acetonitrile in water. Further purification by column chromatography on silica gel, eluting with a mixture of dichloromethane and methanol (49:1 by volume) gave title compound as a yellow solid, 0.021 g.

MS: ESI (+ve) (Method A): 435 (M+H)⁺, Retention time 9.9 min.

¹H NMR (CD₃OD): δ 2.25 (s, 3H), 3.20 (s, 3H), 3.90 (s, 2H), 6.80 (dd, J=1.8, 7.2 Hz, 1H), 7.00 (dd, J=1.8, 8.4 Hz, 1H), 7.15 (d, J=1.8 Hz, 1H), 7.85 (d, J=8.4 Hz, 1H), 7.90 (m, 1H), 8.20 (d, J=7.2 Hz, 1H).

### Example 14

### [7-cyano-1-(4-ethanesulfonylphenylsulfanyl)-2-methylindolizin-3-yl]acetic acid

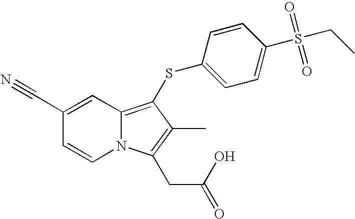

### Preparation 14a

### 1-ethylsulfanyl-4-fluorobenzene

A mixture of diethyl disulfide (11 mL), 4-fluorophenylamine (5.0 g) and dichloromethane (50 mL) at room temperature was treated dropwise with tert-butyl nitrite (7.7 mL) and the resulting mixture stirred at 50° C. for 5 minutes and then at room temperature for 3 hours. The mixture was washed with water and 1.0 M aqueous hydrochloric acid and then dried over magnesium sulfate. The solvent was removed under reduced pressure and the residue purified by column chromatography on silica gel, eluting with cyclohexane to afford title compound, 3.5 g.

¹H NMR (CDCl₃): δ 1.25 (t, J=7.3 Hz, 3H), 2.90 (q, J=7.3 Hz, 2H), 7.00 (m, 2H), 7.35 (m, 2H).

### Preparation 14b

### 1-ethanesulfonyl-4-fluorobenzene

A solution of 1-ethylsulfanyl-4-fluorobenzene (3.5 g) in dichloromethane (20 mL) at room temperature was treated with 3-chloroperoxybenzoic acid (6.0 g) and the resulting mixture was stirred at room temperature overnight. The mixture was washed with saturated aqueous sodium hydrogen carbonate solution, dried over magnesium sulfate and the solvent removed under reduced pressure to afford title compound, 4.3 g.

¹H NMR (DMSO-d6): δ 1.10 (t, J=7.3 Hz, 3H), 3.30 (q, J=7.3 Hz, 2H), 7.50 (m, 2H), 8.00 (m, 2H).

### Preparation 14c

### bis(4-ethanesulfonylbenzene)disulfide

A mixture of 1-ethanesulfonyl-4-fluorobenzene (1.0 g), sodium hydrogensulfide (4.4 g) and 1-methylpyrrolidin-2-one (4.0 mL) was stirred at 80° C. for 2 hours and then at room temperature overnight. The mixture was diluted with water, washed with ethyl acetate and the aqueous phase acidified by the addition of 1.0 M aqueous hydrochloric acid. The mixture was extracted with ethyl acetate and the combined extracts dried over magnesium sulfate and the solvent removed under reduced pressure to afford title compound, 0.80 g.

¹H NMR (DMSO-d6): δ 1.10 (t, J=7.4 Hz, 6H), 3.30 (q, J=7.4 Hz, 4H), 7.80-7.90 (m, 8H).

### Preparation 14d

### [7-cyano-1-(4-ethanesulfonylphenylsulfanyl)-2-methylindolizin-3-yl]acetic acid ethyl ester

The title compound was prepared by the method of Preparation 6d using (7-cyano-2-methylindolizin-3-yl)acetic acid ethyl ester and bis(4-ethanesulfonylbenzene)disulfide.

MS: ESI (+ve) (Method B): 443 (M+H)⁺, Retention time 3.9 min.

### Preparation 14e

### [7-cyano-1-(4-ethanesulfonylphenylsulfanyl)-2-methylindolizin-3-yl]acetic acid

A mixture of [7-cyano-1-(4-ethanesulfonylphenylsulfanyl)-2-methylindolizin-3-yl]acetic acid ethyl ester (0.43 g), tetrahydrofuran (30 mL), water (30 mL) and lithium hydroxide (0.13 g) was stirred at room temperature for 2 hours. The mixture was acidified by the addition of 1.0 M aqueous hydrochloric acid and extracted with ethyl acetate. The combined extracts were dried over magnesium sulfate and the solvent removed under reduced pressure. The residue was purified by preparative reverse-phase HPLC using a gradient of acetonitrile in water. Further purification by column chromatography on silica gel, eluting with a mixture of dichloromethane and methanol (49:1 by volume) gave title compound as a yellow solid, 0.044 g.

MS: ESI (+ve) (Method A): 415 (M+H)⁺, Retention time 9.6 min.

¹H NMR (CD₃OD): δ 1.15 (t, J=7.4 Hz, 3H), 2.25 (s, 3H), 3.10 (q, J=7.4 Hz, 2H), 3.85 (s, 2H), 6.80 (d, J=1.8, 7.4 Hz, 1H), 7.15 (d, J=8.6 Hz, 2H), 7.65 (d, J=8.6 Hz, 2H), 7.85 (m, 1H), 8.20 (d, J=7.4 Hz, 1H).

### Example 15

### [1-(4-chlorobenzyl)-7-cyano-2-methylindolizin-3-yl]acetic acid

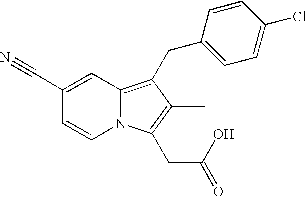

### Preparation 15a

### [1-(4-chlorobenzyl)-7-cyano-2-methylindolizin-3-yl]acetic acid ethyl ester

A mixture of (7-cyano-2-methylindolizin-3-yl)acetic acid ethyl ester (0.15 g), 4-chlorobenzaldehyde (0.087 g) and 1,2-dichloroethane (2.0 mL) at 0° C. was treated dropwise with triethylsilane (0.49 mL) followed by trifluoroacetic acid (0.14 mL) and the resulting mixture was stirred at 0° C. for 10 minutes and then at room temperature for 18 hours. The mixture was concentrated under reduced pressure and the residue partitioned between ethyl acetate and saturated aqueous sodium hydrogen carbonate solution. The organic phase was washed with water and saturated aqueous sodium chloride solution and then dried over magnesium sulfate. The solvent was removed under reduced pressure and the residue purified by column chromatography on silica gel, eluting with a mixture of cyclohexane and ethyl acetate (1:0 to 7:3 by volume) to afford title compound as a yellow solid, 0.19 g.

MS: ESI (+ve) (Method B): 367 (M+H)⁺, Retention time 4.3 min.

### Preparation 15b

### [1-(4-chlorobenzyl)-7-cyano-2-methylindolizin-3-yl]acetic acid

A mixture of [1-(4-chlorobenzyl)-7-cyano-2-methylindolizin-3-yl]acetic acid ethyl ester (0.19 g), methanol (8.0 mL) and 1.0 M aqueous sodium hydroxide solution (3.5 mL) was stirred at room temperature for 2 hours. The mixture was concentrated under reduced pressure, diluted with water and acidified by the addition of glacial acetic acid. The mixture was extracted with dichloromethane and the combined extracts dried over magnesium sulfate. The solvent was removed under reduced pressure and the residue purified by preparative reverse-phase HPLC using a gradient over 35 minutes of acetonitrile in water (40% to 80% of organic modifier) to afford title compound as a yellow solid, 0.064 g.

MS: ESI (+ve) (Method B): 339 (M+H)⁺, Retention time 3.7 min.

¹H NMR (CDCl₃): δ 2.15 (s, 3H), 3.90 (s, 2H), 4.05 (s, 2H), 6.55 (dd, J=1.4, 7.3 Hz, 1H), 7.00 (d, J=8.3 Hz, 2H), 7.15 (d, J=8.3 Hz, 2H), 7.65 (s, 1H), 7.75 (d, J=7.3 Hz, 1H).

### Example 16

### [1-(2-chloro-4-methanesulfonylphenylsulfanyl)-7-cyano-2-methylindolizin-3-yl]acetic acid

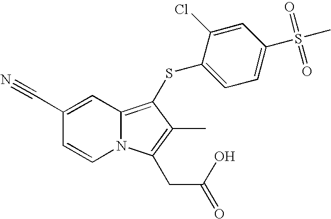

### Preparation 16a

### 2-chloro-1-fluoro-4-methanesulfonylbenzene

A solution of sodium nitrite (5.7 g) in water (15 mL) was added dropwise over a period 15 minutes to a mixture of 2-fluoro-5-methanesulfonylphenylamine (15 g), concentrated hydrochloric acid (30 mL) and water (90 mL) at 0-10° C. The solution was added to a mixture of copper chloride(I) (11.8 g) and concentrated hydrochloric acid (30 mL) at 0° C. and the resulting mixture heated at 40° C. for 10 minutes. The mixture was cooled to room temperature, filtered and the filtrate extracted with dichloromethane. The combined extracts were dried over magnesium sulfate and the solvent removed under reduced pressure. Purification of the residue by column chromatography on silica gel, eluting with a mixture of toluene, dichloromethane and ethyl acetate (2:1:0 to 0:1:0 to 0:50:1 by volume) gave title compound as a white solid, 3.7 g.

¹H NMR (CDCl₃): δ 3.05 (s, 3H), 7.35 (t, J=8.4 Hz, 1H), 7.85 (m, 1H), 8.05 (dd, J=2.2, 6.6 Hz, 1H).

### Preparation 16b

### bis(2-chloro-4-methanesulfonylbenzene)disulfide

A mixture of 2-chloro-1-fluoro-4-methanesulfonylbenzene (1.0 g), sodium hydrogensulfide (3.6 g) and 1-methylpyrrolidin-2-one (5.0 mL) was stirred at 80° C. for 90 minutes and then at room temperature for 4 hours. The mixture was diluted with water (40 mL) and acidified by the addition of concentrated hydrochloric acid. The resulting precipitate was collected by filtration, washed with water and ethanol and then crystallised from toluene to give title compound as a white solid, 0.61 g.

¹H NMR (DMSO-d6): δ 3.30 (s, 6H), 7.80 (d, J=8.3 Hz, 2H), 7.90 (dd, J=1.5, 8.3 Hz, 2H), 8.10 (br s, 2H).

MS: ESI (+ve) (Method B): 443 (M+H)⁺, Retention time 3.8 min.

### Preparation 16c

### [1-(2-chloro-4-methanesulfonylphenylsulfanyl)-7-cyano-2-methylindolizin-3-yl]acetic acid ethyl ester

The title compound was prepared by the method of Preparation 6d using (7-cyano-2-methylindolizin-3-yl)acetic acid ethyl ester and bis(2-chloro-4-methanesulfonylbenzene)disulfide.

MS: ESI (+ve) (Method B): 463 (M+H)⁺, Retention time 3.7 min.

### Preparation 16d

### [1-(2-chloro-4-methanesulfonylphenylsulfanyl)-7-cyano-2-methylindolizin-3-yl]acetic acid

A mixture of [1-(2-chloro-4-methanesulfonylphenylsulfanyl)-7-cyano-2-methylindolizin-3-yl]acetic acid ethyl ester (0.027 g), ethanol (5.0 mL), water (0.5 mL) and 5.0 M aqueous lithium hydroxide solution (0.25 mL) was stirred at room temperature for 1 hour. The mixture was acidified by the addition of glacial acetic acid and concentrated under reduced pressure. The residue was partitioned between ethyl acetate and water and the organic phase dried over magnesium sulfate. The solvent was removed under reduced pressure and the residue purified by preparative reverse-phase HPLC using a gradient of acetonitrile in water to afford title compound as a yellow solid, 0.037 g.

MS: ESI (+ve) (Method A): 435 (M+H)⁺, Retention time 10.6 min.

¹H NMR (DMSO-d6): δ 2.15 (s, 3H), 3.15 (s, 3H), 4.10 (s, 2H), 6.45 (d, J=8.4 Hz, 1H), 7.00 (dd, J=1.8, 7.3 Hz, 1H), 7.55 (dd, J=2.0, 8.4 Hz, 1H), 7.90 (d, J=1.8, 8.4 Hz, 1H), 8.05 (m, 1H), 8.40 (dd, J=0.8, 7.3 Hz, 1H), 12.70 (br s, 1H).

### Example 17

### [7-cyano-1-(4-methanesulfonyl-2-trifluoromethylphenylsulfanyl)-2-methylindolizin-3-yl]acetic acid

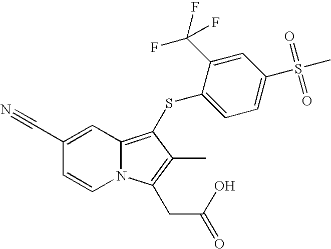

### Preparation 17a

### 1-fluoro-4-methylsulfanyl-2-trifluoromethylbenzene

A mixture of dimethyl disulfide (2.0 mL), 4-fluoro-3-trifluoromethylphenylamine (2.0 g) and acetonitrile (50 mL) at room temperature was treated dropwise with isopentyl nitrite (2.0 mL) and the resulting mixture was stirred at 60° C. for 16 hours. The mixture was cooled to room temperature, diluted with water and extracted with ethyl acetate. The combined extracts were dried over magnesium sulfate and the solvent was removed under reduced pressure to afford title compound, 0.67 g.

¹H NMR (CDCl₃): δ 2.50 (s, 3H), 7.15 (m, 1H), 7.40-7.50 (m, 2H).

### Preparation 17b

### 1-fluoro-4-methanesulfonyl-2-trifluoromethylbenzene

3-Chloroperoxybenzoic acid (2.4 g) was added portionwise to a solution of 1-fluoro-4-methylsulfanyl-2-trifluoromethylbenzene (1.3 g) in dichloromethane (50 mL) at room temperature and the resulting mixture was stirred at room temperature for 3 hours. The mixture was filtered and the filtrated washed with saturated aqueous sodium thiosulfate solution and saturated aqueous sodium chloride solution and then dried over magnesium sulfate. The solvent was removed under reduced pressure to afford title compound, 1.5 g.

¹H NMR (CDCl₃): δ 3.1 (s, 3H), 7.45 (t, J=9.3 Hz, 1H), 8.20 (m, 1H), 8.25 (m, 1H).

### Preparation 17c

### bis(2-trifluoromethyl-4-methanesulfonylbenzene)disulfide

A mixture of 1-fluoro-4-methanesulfonyl-2-trifluoromethylbenzene (1.4 g), sodium hydrogensulfide (4.6 g) and 1-methylpyrrolidin-2-one (6.0 mL) was stirred at 80° C. for 2 hours and then at room temperature overnight. The mixture was diluted with water, washed with ethyl acetate and the aqueous phase acidified by the addition of concentrated hydrochloric acid. The mixture was extracted with ethyl acetate and the combined extracts dried over magnesium sulfate and the solvent removed under reduced pressure. The residue was triturated with ethyl acetate to afford title compound, 0.94 g.

### Preparation 17d

### [7-cyano-1-(4-methanesulfonyl-2-trifluoromethylphenylsulfanyl)-2-methylindolizin-3-yl]acetic acid ethyl ester

The title compound was prepared by the method of Preparation 6d using (7-cyano-2-methylindolizin-3-yl)acetic acid ethyl ester and bis(2-trifluoromethyl-4-methanesulfonylbenzene)disulfide.

MS: ESI (+ve) (Method B): 497 (M+H)⁺, Retention time 3.7 min.

### Preparation 17e

### [7-cyano-1-(4-methanesulfonyl-2-trifluoromethylphenylsulfanyl)-2-methylindolizin-3-yl]acetic acid

A mixture of [7-cyano-1-(4-methanesulfonyl-2-trifluoromethylphenylsulfanyl)-2-methylindolizin-3-yl]acetic acid ethyl ester (0.029 g), ethanol (5.0 mL), water (0.5 mL) and 5.0 M aqueous lithium hydroxide solution (0.25 mL) was stirred at room temperature for 1 hour. The mixture was acidified by the addition of glacial acetic acid and concentrated under reduced pressure. The residue was partitioned between ethyl acetate and water and the organic phase dried over magnesium sulfate. The solvent was removed under reduced pressure and the residue purified by preparative reverse-phase HPLC using a gradient of acetonitrile in water to afford title compound as a yellow solid, 0.005 g.

MS: ESI (+ve) (Method B): 469 (M+H)⁺, Retention time 3.2 min.

### Example 18

### [7-chloro-1-(4-methanesulfonylphenylsulfanyl)-2-methylindolizin-3-yl]acetic acid

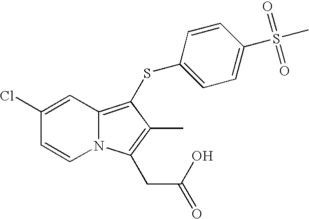

### Preparation 18a

### 7-chloro-2-methylindolizine

A mixture of 1-bromopropan-2-one (4.2 g), 4-chloro-2-methylpyridine (5.0 g) and acetonitrile (30 mL) was heated at reflux for 1 hour. The mixture was cooled to room temperature and concentrated under reduced pressure. The residue was diluted with water (100 mL), treated with sodium hydrogen carbonate (2.6 g) and then distilled under atmospheric pressure. The distillate was extracted with ethyl acetate and the combined extracts dried over magnesium sulfate. The solvent was removed under reduced pressure to afford title compound as a pale yellow solid, 1.6 g.

¹H NMR (CDCl₃): δ 2.30 (s, 3H), 6.20 (br s, 1H), 6.35 (dd, J=2.2, 7.3 Hz, 1H), 7.05 (m, 1H), 7.20 (d, J=2.2 Hz, 1H), 7.70 (dt, J=0.9, 7.3 Hz, 1H).

### Preparation 18b

### (7-chloro-2-methylindolizin-3-yl)acetic acid ethyl ester

To a mixture of 7-chloro-2-methylindolizine (2.3 g), copper bronze (2.2 g) and toluene (50 mL) at reflux was added dropwise a solution of ethyl diazoacetate (3.6 mL) in toluene (20 mL). The resulting mixture was heated at reflux for 1 hour and then concentrated removed under reduced pressure. The residue was purified by column chromatography on silica gel, eluting with a mixture of cyclohexane and ethyl acetate (1:0 to 7:3 by volume) to afford title compound, 1.4 g.

MS: ESI (+ve) (Method B): 252 (M+H)⁺, Retention time 3.6 min.

### Preparation 18c

### [7-chloro-1-(4-methanesulfonylphenylsulfanyl)-2-methylindolizin-3-yl]acetic acid ethyl ester

The title compound was prepared by the method of Preparation 6d using (7-chloro-2-methylindolizin-3-yl)acetic acid ethyl ester and bis[4-(methylsulfonyl)phenyl]disulfide

MS: ESI (+ve) (Method B): 438 (M+H)⁺, Retention time 3.7 min.

### Preparation 18d

### [7-chloro-1-(4-methanesulfonylphenylsulfanyl)-2-methylindolizin-3-yl]acetic acid

A mixture of [7-chloro-1-(4-methanesulfonylphenylsulfanyl)-2-methylindolizin-3-yl]acetic acid ethyl ester (0.080 g), ethanol (5.0 mL), water (0.5 mL) and 5.0 M aqueous lithium hydroxide solution (0.25 mL) was stirred at room temperature for 1 hour. The mixture was acidified by the addition of glacial acetic acid and then concentrated under reduced pressure. The residue was partitioned between ethyl acetate and water and the organic phase dried over magnesium sulfate. The solvent was removed under reduced pressure and the residue purified by preparative reverse-phase HPLC using a gradient of acetonitrile in water to afford title compound as a blue/green solid, 0.004 g.

MS: ESI (+ve) (Method B): 410 (M+H)⁺, Retention time 3.2 min.

¹H NMR (DMSO-d6): δ 2.10 (s, 3H), 3.10 (s, 3H), 4.00 (s, 2H), 6.80 (dd, J=2.2, 7.4 Hz, 1H), 7.05 (d, J=8.6 Hz, 2H), 7.40 (d, J=2.2 Hz, 1H), 7.70 (d, J=8.6 Hz, 2H), 8.25 (d, J=7.4 Hz, 1H).

### Example 19

### [7-chloro-1-(2-fluoro-4-methanesulfonylphenylsulfanyl)-2-methylindolizin-3-yl]acetic acid

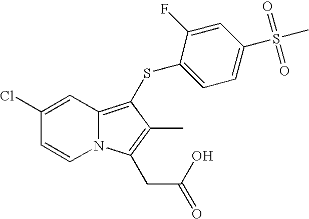

### Preparation 19a

### 1,2-difluoro-4-methanesulfonylbenzene

3-Chloroperoxybenzoic acid (19 g) was added portionwise to a solution of 1,2-difluoro-4-methylsulfanylbenzene (5.4 g) in dichloromethane (100 mL) at room temperature and the resulting mixture was stirred at room temperature overnight. The mixture was washed with saturated aqueous sodium thiosulfate solution and saturated aqueous sodium hydrogen carbonate solution and then dried over magnesium sulfate. The solvent was removed under reduced pressure to afford title compound as a white solid, 5.5 g.

¹H NMR (CDCl₃): δ 3.05 (s, 3H), 7.40 (m, 1H), 7.75-7.85 (m, 2H).

### Preparation 19b

### bis(2-fluoro-4-methanesulfonylbenzene)disulfide

A mixture of 1,2-difluoro-4-methanesulfonylbenzene (2.8 g), sodium hydrogensulfide (11 g) and 1-methylpyrrolidin-2-one (30 mL) was stirred at 80° C. for 90 minutes and then at room temperature overnight. The mixture was diluted with water (40 mL), acidified by the addition of concentrated hydrochloric acid and then extracted with ethyl acetate. The combined extracts were dried over magnesium sulfate and the solvent removed under reduced pressure. The residue was crystallised from toluene to afford title compound as cream crystals, 1.4 g.

MS: ESI (+ve) (Method B): 411 (M+H)⁺, Retention time 3.1 min.

### Preparation 19c

### [7-chloro-1-(2-fluoro-4-methanesulfonylphenylsulfanyl)-2-methylindolizin-3-yl]acetic acid ethyl ester

The title compound was prepared by the method of Preparation 6d using (7-chloro-2-methylindolizin-3-yl)acetic acid ethyl ester and bis(2-fluoro-4-methanesulfonylbenzene)disulfide.

MS: ESI (+ve) (Method B): 456 (M+H)⁺, Retention time 3.8 min.

### Preparation 19d

### [7-chloro-1-(2-fluoro-4-methanesulfonylphenylsulfanyl)-2-methylindolizin-3-yl]acetic acid

A mixture of [7-chloro-1-(2-fluoro-4-methanesulfonylphenylsulfanyl)-2-methylindolizin-3-yl]acetic acid ethyl ester (0.082 g), ethanol (5.0 mL), water (0.5 mL) and 5.0 M aqueous lithium hydroxide solution (0.25 mL) was stirred at room temperature for 1 hour. The mixture was acidified by the addition of glacial acetic acid and then concentrated under reduced pressure. The residue was partitioned between ethyl acetate and water and the organic phase dried over magnesium sulfate. The solvent was removed under reduced pressure and the residue purified by preparative reverse-phase HPLC using a gradient of acetonitrile in water to afford title compound as a blue/green solid, 0.019 g.

MS: ESI (+ve) (Method B): 428 (M+H)⁺, Retention time 3.7 min.

¹H NMR (DMSO-d6): δ 2.10 (s, 3H), 3.15 (s, 3H), 4.00 9s, 2H), 8.50 (t, J=7.8 Hz, 1H), 6.80 (dd, J=2.2, 7.4 Hz, 1H), 7.45 (m, 1H), 7.50 (dd, J=1.8, 8.3 Hz, 1H), 7.70 (dd, J=1.8, 7.8 Hz, 1H), 8.25 (d, J=7.4 Hz, 1H), 12.60 (br s, 1H).

### Example 20

### [7-cyano-1-(4-methanesulfonylbenzyl)-2-methylindolizin-3-yl]acetic acid

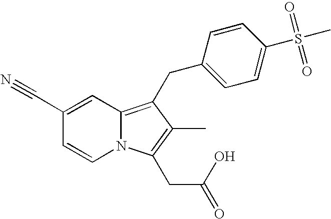

### Preparation 20a

### [7-cyano-1-(4-methanesulfonylbenzyl)-2-methylindolizin-3-yl]acetic acid ethyl ester

The title compound was prepared by the method of Preparation 15a using (7-cyano-2-methylindolizin-3-yl)acetic acid ethyl ester and 4-methanesulfonylbenzaldehyde.

MS: ESI (+ve) (Method B): 411 (M+H)⁺, Retention time 3.6 min.

### Preparation 20b

### [7-cyano-1-(4-methanesulfonylbenzyl)-2-methylindolizin-3-yl]acetic acid

The title compound was prepared by the method of Preparation 15b using [7-cyano-1-(4-methanesulfonylbenzyl)-2-methylindolizin-3-yl]acetic acid ethyl ester.

MS: ESI (+ve) (Method B): 383 (M+H)⁺, Retention time 3.0 min.

### Example 21

### [7-chloro-1-(6-fluoroquinolin-2-ylmethyl)-2-methylindolizin-3-yl]acetic acid

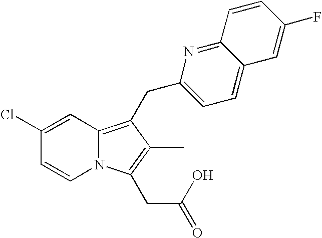

### Preparation 21a

### 6-fluoroquinoline-2-carbaldehyde

Prepared as described in WO2005044260

### Preparation 21b

### [7-chloro-1-(6-fluoroquinolin-2-ylmethyl)-2-methylindolizin-3-yl]acetic acid ethyl ester

The title compound was prepared by the method of Preparation 15a using (7-chloro-2-methylindolizin-3-yl)acetic acid ethyl ester and 6-fluoroquinoline-2-carbaldehyde.

MS: ESI (+ve) (Method B): 411 (M+H)⁺, Retention time 3.7 min.

### Preparation 21c

### [7-chloro-1-(6-fluoroquinolin-2-ylmethyl)-2-methylindolizin-3-yl]acetic acid

A mixture of [7-chloro-1-(6-fluoroquinolin-2-ylmethyl)-2-methylindolizin-3-yl]acetic acid ethyl ester (0.52 g), tetrahydrofuran (5.0 mL) and 1.0 M aqueous lithium hydroxide solution (2.0 mL) was stirred at room temperature for 2 hours. The mixture was concentrated under reduced pressure, acidified by the addition sodium dihydrogenphosphate monohydrate and then extracted with ethyl acetate. The combined extracts were dried over magnesium sulfate and the solvent removed under reduced pressure. The residue was triturated with a mixture of acetonitrile and water to afford title compound as a yellow solid, 0.36 g.

MS: ESI (+ve) (Method B): 383 (M+H)⁺, Retention time 3.2 min.

### Example 22

### [1-(4-methanesulfonylphenylsulfanyl)-2-methyl-7-trifluoromethylindolizin-3-yl]acetic acid

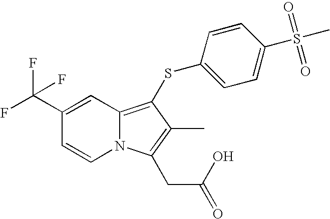

### Preparation 22a

### 2-methyl-4-trifluoromethylpyridine

A mixture of 2-chloro-4-trifluoromethylpyridine (16 g), tetrakis(triphenylphosphine) palladium(0) (2.0 g), trimethylaluminum (2.0 M in hexanes, 48 mL) and 1,4-dioxane (300 mL) was heated at reflux for 2.5 hours. The mixture was cooled to 0° C., acidified by the addition 1.0 M aqueous hydrochloric acid and the organic phase dried over magnesium sulfate and then carefully concentrated to low bulk under atmospheric pressure. The residue was purified by column chromatography on silica gel to afford title compound, 1.5 g.

¹H NMR (CDCl₃): δ 2.65 (s, 3H), 7.30 (m, 1H), 7.35 (s, 1H), 8.70 (d, J=5.2 Hz, 1H).

### Preparation 22b

### 2-methyl-7-trifluoromethylindolizine

A mixture of 1-bromopropan-2-one (3.5 g), acetonitrile (25 mL), sodium hydrogen carbonate (1.1 g) and 2-methyl-4-trifluoromethylpyridine (1.5 g) was heated at reflux overnight. The mixture was diluted with water, extracted with dichloromethane and the combined extracts dried over sodium sulfate. The solvent was removed under reduced pressure and the residue purified by column chromatography on silica gel, eluting with a mixture of cyclohexane and ethyl acetate (9:1 by volume) to afford title compound, 0.52 g.

¹H NMR (CDCl₃): δ 2.35 (s, 3H), 6.45 (s, 1H), 6.50 (dd, J=1.9, 7.3 Hz, 1H), 7.20 (m, 1H), 7.55 (m, 1H), 7.85 (m, 1H).

### Preparation 22c

### (2-methyl-7-trifluoromethylindolizin-3-yl)acetic acid ethyl ester

The title compound was prepared by the method of Preparation 18b using 2-methyl-7-trifluoromethylindolizine.

¹H NMR (CDCl₃): δ 1.25 (t, J=7.2 Hz, 3H), 2.35 (s, 3H), 3.85 (s, 2H), 4.15 (q, J=7.2 Hz, 2H), 6.50 (s, 1H), 6.65 (dd, J=1.9, 7.4 Hz, 1H), 7.60 (m, 1H), 7.90, (m, 1H).

### Preparation 22d

### [1-(4-methanesulfonylphenylsulfanyl)-2-methyl-7-trifluoromethylindolizin-3-yl]acetic acid ethyl ester

The title compound was prepared by the method of Preparation 6d using (2-methyl-7-trifluoromethylindolizin-3-yl)acetic acid ethyl ester and bis[4-(methylsulfonyl)phenyl]disulfide.

MS: ESI (+ve) (Method B): 472 (M+H)⁺, Retention time 4.2 min.

### Preparation 22e

### [1-(4-methanesulfonylphenylsulfanyl)-2-methyl-7-trifluoromethylindolizin-3-yl]acetic acid

The title compound was prepared by the method of Preparation 14e using [1-(4-methanesulfonylphenylsulfanyl)-2-methyl-7-trifluoromethylindolizin-3-yl]acetic acid ethyl ester.

MS: ESI (+ve) (Method B): 444 (M+H)⁺, Retention time 3.7 min.

### Biological Results:

The compounds of the Examples 1-14 above were tested in the CRTH2 Radioligand Binding assay described above; the compounds had Kᵢ values of less than <1 μM in the binding assay. Examples 1-4 were tested in the GTPγS functional assay, and showed IC₅₀ values of <1 μM.

1. A compound of formula (I) or a salt, N-oxide, hydrate or solvate thereof: 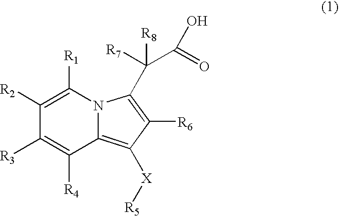
wherein
R₁, R₂. R₃ and R₄ each independently are hydrogen, C₁-C₆alkyl, fully or partially fluorinated C₁-C₆alkyl, halo, —S(O)ₙR₁₀, —SO₂N(R₁₀)₂, —N(R₁₀)₂, —C(O)N(R₁₀)₂, —NR₁₀C(O)R₉, —CO₂R₁₀, —C(O)R₉, —NO₂, —CN or —OR₁₁;
wherein each R₉ is independently C₁-C₆alkyl, aryl, or heteroaryl;
R₁₀ is independently hydrogen, C₁-C₆alkyl, aryl, or heteroaryl;
R₁₁ is hydrogen, C₁-C₆alkyl, fully or partially fluorinated C₁-C₆alkyl or a group —SO₂R₁₀;
n is 0, 1 or 2;
R₅ is C₁-C₆alkyl, fully or partially fluorinated C₁-C₆alkyl, C₁-C₆alkenyl, C₁-C₆alkynyl, optionally substituted aryl, or optionally substituted heteroaryl;
R₆ is hydrogen, C₁-C₆alkyl or fully or partially fluorinated C₁-C₆alkyl;
R₇ and R₈ are independently hydrogen or C₁-C₆alkyl, or R₇ and R₈ together with the atom to which they are attached form a cycloalkyl group; and
X is —CHR₆—, —S(O)ₙ—, —NR₆SO₂— or —SO₂NR₆— wherein n is 0, 1 or 2.

2. The compound as claimed in claim 1 wherein R₁, R₂, R₃ and R₄ are independently selected from hydrogen, methyl, ethyl, trifluoromethyl, fluoro, chloro, bromo, —NO₂, —CN, —SO₂R₉, —SO₂N(R₁₀)₂, —C(O)N(R₁₀)₂, —NR₁₀C(O)R₉, —CO₂R₁₀, and
—C(O)R₉.

3. The compound as claimed in claim 2 wherein any R₉ is selected from methyl, ethyl, or phenyl; any R₁₀ is selected from hydrogen, methyl, ethyl, or phenyl; and any R₁₁ is selected from methyl, trifluoromethyl, ethyl, phenyl, —SO₂H and —SO₂CH₃.

4. The compound as claimed in claim 1 wherein R₁, R₂, R₃ and R₄ are independently selected from hydrogen, chloro, fluoro, cyano, methyl, and trifluoromethyl.

5. The compound as claimed in claim 1 wherein two of R₁, R₂, R₃ and R₄ are hydrogen, while the others are independently selected from hydrogen, chloro, fluoro, cyano, methyl, and trifluoromethyl.

6. The compound as claimed in claim 1 wherein R₅ is methyl, ethyl, n- or iso-propyl, trifluoromethyl, allyl, optionally substituted phenyl or naphthyl; optionally substituted monocyclic heteroaryl having 5 or 6 ring atoms; or optionally substituted bicyclic heteroaryl having 8 to 10 ring atoms.

7. The compound as claimed in claim 1 wherein R₅ is optionally substituted pyridyl, pyrimidinyl, furyl, thienyl, imidazolyl, oxazolyl, isoxazolyl, or pyrrolyl, quinolinyl, indolyl, or benzimidazolyl.

8. The compound as claimed in claim 1 wherein R₅ is optionally substituted phenyl.

9. The compound as claimed in claim 6 wherein the said optional substituents are selected from chloro, fluoro, methylsulfonyl, ethylsulfonyl, carbamate, methylcarbamate, methylaminosulfonyl, ethylaminosulfonyl, methylsulfonylamino, ethylsulfonylamino, morpholin-1-ylsulfonyl, piperidin-1-ylsulfonyl, piperazin-1-ylsulfonyl, 4-methylpiperazin-1-ylsulfonyl, and tetrahydropyrrole-1 ylsulfonyl.

10. The compound as claimed in claim 1 wherein the divalent radical —X— is —CH₂—, —S(O)ₙ—, —NHSO₂— or —SO₂NH—.

11. The compound as claimed in claim 1 wherein the divalent radical —X— is —S— or —SO₂—.

12. The compound as claimed in claim 1 wherein R₆ is hydrogen, ethyl or trifluoromethyl.

13. The compound as claimed in claim 1 wherein R₆ is methyl.

14. The compound as claimed in claim 1 wherein R₇ and R₈ are each hydrogen.

15. The compound as claimed in claim 1 wherein one of R₇ and R₈ is methyl while the other is hydrogen.

16. The compound as claimed in claim 1 wherein R₇ and R₈ taken together with the carbon atom to which they are attached form a cyclopropyl, cyclopentyl or cyclohexyl ring.

17. The compound as claimed in claim 1 wherein R₁, R₂, R₃ and R₄ are independently selected from hydrogen, chloro, fluoro, cyano, methyl, trifluoromethyl, and di- or trifluoromethoxy; X is —S—, —SO₂—, or —CH₂—; R₅ is optionally substituted phenyl or optionally substituted heteroaryl; R₆ is methyl, and R₇ and R₈ are hydrogen.

18. The compound as claimed in claim 17 wherein R₅ is phenyl or phenyl substituted by one or two substituents selected from chloro, fluoro, trifluoromethyl, methylsulfonyl, ethylsulfonyl, carbamate, methylcarbamate, methylaminosulfonyl, ethylaminosulfonyl, methylsulfonylamino, ethylsulfonylamino, morpholin-1-ylsulfonyl, piperidin-1-ylsulfonyl, piperazin-1-ylsulfonyl, 4-methylpiperazin-1-ylsulfonyl, and tetrahydropyrrole-1 ylsulfonyl.

19. An ester prodrug of a compound as claimed in claim 1 wherein the carboxylic acid group attached to —C(R₇)(R₈)— is esterified as an ester which is hydrolysed in vivo to release the carboxylic acid.

20. (canceled)

21. A pharmaceutical composition comprising a compound of formula (I) or a salt, N-oxide, hydrate or solvate thereof: 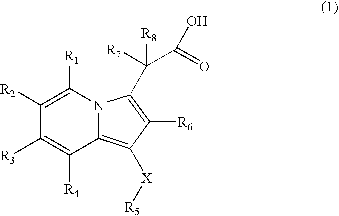
wherein
R₁, R₂. R₃ and R₄ each independently are hydrogen, C₁-C₆alkyl, fully or partially fluorinated C₁-C₆alkyl, halo, —S(O)ₙR₁₀, —SO₂N(R₁₀)₂, —N(R₁₀)₂, —C(O)N(R₁₀)₂, —NR₁₀C(O)R₉, —CO₂R₁₀, —C(O)R₉, —NO₂, —CN or —OR₁₁;
wherein each R₉ is independently C₁-C₆alkyl, aryl, or heteroaryl;
R₁₀ is independently hydrogen, C₁-C₆alkyl, aryl, or heteroaryl;
R₁₁ is hydrogen, C₁-C₆alkyl, fully or partially fluorinated C₁-C₆alkyl or a group —SO₂R₁₀;
n is 0, 1 or 2;
R₅ is C₁-C₆alkyl, fully or partially fluorinated C₁-C₆ alkyl, C₁-C₆alkenyl, C₁-C₆alkynyl, optionally substituted aryl, or optionally substituted heteroaryl;
R₆ is hydrogen, C₁-C₆alkyl or fully or partially fluorinated C₁-C₆alkyl;
R₇ and R₈ are independently hydrogen or C₁-C₆alkyl, or R₇ and R₈ together with the atom to which they are attached form a cycloalkyl group; and
X is —CHR₆—, —S(O)ₙ, —NR₆SO₂— or —SO₂NR₆— wherein n is 0, 1 or 2;
and a pharmaceutically acceptable carrier.

22. A method of treatment of asthma, chronic obstructive pulmonary disease, rhinitis, allergic airway syndrome, or allergic rhinobronchitis, comprising administering to a patient suffering such disease an effective amount of a compound of formula (1) or a salt, N-oxide, hydrate or solvate thereof: 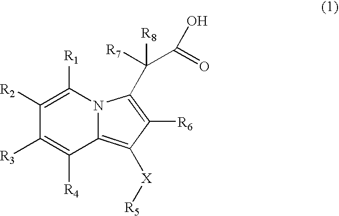
wherein
R₁, R₂. R₃ and R₄ each independently are hydrogen, C₁-C₆alkyl, fully or partially fluorinated C₁-C₆alkyl, halo, —S(O)ₙR₁₀, —SO₂N(R₁₀)₂, —N(R₁₀)₂, —C(O)N(R₁₀)₂, —NR₁₀C(O)R₉, —CO₂R₁₀, —C(O)R₉, —NO₂, —CN or —OR₁₁;
wherein each R₉ is independently C₁-C₆alkyl, aryl, or heteroaryl;
R₁₀ is independently hydrogen, C₁-C₆alkyl, aryl, or heteroaryl;
R₁₁ is hydrogen, C₁-C₆alkyl, fully or partially fluorinated C₁-C₆alkyl or a group —SO₂R₁₀;
n is 0, 1 or 2;
R₅ is C₁-C₆alkyl, fully or partially fluorinated C₁-C₆alkyl, C₁-C₆alkenyl, C₁-C₆alkynyl, optionally substituted aryl, or optionally substituted heteroaryl;
R₆ is hydrogen, C₁-C₆alkyl or fully or partially fluorinated C₁-C₆alkyl;
R₇ and R₈ are independently hydrogen or C₁-C₆alkyl, or R₇ and R₈ together with the atom to which they are attached form a cycloalkyl group; and
X is —CHR₆—, —S(O)ₙ—, —NR₆SO₂— or —SO₂NR₆— wherein n is 0, 1 or 2.

23. (canceled)
